SEQUENCE LISTING (118 sequences)

SEQ ID NO: 1 - DNA, 23 bp, Artificial
  forward pcr primer
tctccttggatatacttgtgtga

SEQ ID NO: 2 - DNA, 22 bp, Artificial
  reverse pcr primer
cacctattcaccagatttcgta

SEQ ID NO: 3 - DNA, 24 bp, Artificial
  forward pcr primer
ctcctctaaagatgaaaagtcttg

SEQ ID NO: 4 - DNA, 24 bp, Artificial
  reverse pcr primer
tttatcccttacttgtaccagctc

SEQ ID NO: 5 - DNA, 24 bp, Artificial
  forward pcr primer
gaaacatttatgaacctgagaaga

SEQ ID NO: 6 - DNA, 22 bp, Artificial
  reverse pcr primer
ttccagaatagggaagctagag

SEQ ID NO: 7 - DNA, 22 bp, Artificial
  forward pcr primer
gaacagaactgaaactgactcg

SEQ ID NO: 8 - DNA, 22 bp, Artificial
  reverse pcr primer
tctatcttttcgcacatttttg

SEQ ID NO: 9 - DNA, 24 bp, Artificial
  forward pcr primer
cataaaacaagcatctattgaaaa

SEQ ID NO: 10 - DNA, 22 bp, Artificial
  reverse pcr primer
gagacatggacaccaaattaag

SEQ ID NO: 11 - DNA, 22 bp, Artificial
  forward pcr primer
atgatgggttttatttccagac

SEQ ID NO: 12 - DNA, 22 bp, Artificial
  reverse pcr primer
atttgggtagtgtgaagggttc

SEQ ID NO: 13 - DNA, 22 bp, Artificial
  forward pcr primer
ctttcaaattcagattgagcat

SEQ ID NO: 14 - DNA, 23 bp, Artificial
  reverse pcr primer
tccagaaacagaatataaagcaa

SEQ ID NO: 15 - DNA, 21 bp, Artificial
  forward pcr primer
gatgaccaggaaatagagagg

SEQ ID NO: 16 - DNA, 22 bp, Artificial
  reverse pcr primer
gcaatctatgatgggacagtct

SEQ ID NO: 17 - DNA, 22 bp, Artificial
  forward pcr primer
agctgtgtctgtaaactgatgg

SEQ ID NO: 18 - DNA, 23 bp, Artificial
  reverse pcr primer
ctcagaatctggtactaaggaca

SEQ ID NO: 19 - DNA, 21 bp, Artificial
  forward pcr primer
gggaggaataggtgaagatgt

SEQ ID NO: 20 - DNA, 22 bp, Artificial
  reverse pcr primer
catgctcacaatttattcattt

SEQ ID NO: 21 - DNA, 22 bp, Artificial
  forward pcr primer
ccaaagatatagcaattttgga

SEQ ID NO: 22 - DNA, 22 bp, Artificial
  reverse pcr primer
aatgcaatagacaggacttcaa

SEQ ID NO: 23 - DNA, 22 bp, Artificial
  forward pcr primer
aaatatttcacaggcaggagtc

SEQ ID NO: 24 - DNA, 22 bp, Artificial
  reverse pcr primer
cctgttgttaaaatggaaatga

SEQ ID NO: 25 - DNA, 22 bp, Artificial
  forward pcr primer
gtgaaattgtctgccattctta

SEQ ID NO: 26 - DNA, 22 bp, Artificial
  reverse pcr primer
actgggattcacttactgaaca

SEQ ID NO: 27 - DNA, 23 bp, Artificial
  forward pcr primer
gggtataagcagcatattctcaa

SEQ ID NO: 28 - DNA, 22 bp, Artificial
  reverse pcr primer
ccgcaaataagcttttaagaca

SEQ ID NO: 29 - DNA, 23 bp, Artificial
  forward pcr primer
cctatatgtcacagaagtgatcc

SEQ ID NO: 30 - DNA, 22 bp, Artificial
  reverse pcr primer
tttttctggctaagtccttttg

SEQ ID NO: 31 - DNA, 22 bp, Artificial
  forward pcr primer
tggtaagtacatgggtgtttct

SEQ ID NO: 32 - DNA, 22 bp, Artificial
  reverse pcr primer
tggtaagtacatgggtgtttct

SEQ ID NO: 33 - DNA, 50 bp, Artificial
  ASPE primer
tactataatctcacttctatttcaagatccttacccctaaatataaaaag

SEQ ID NO: 34 - DNA, 48 bp, Artificial
  ASPE primer
ctatcatttatctctttctcaattagatccttacccctaaatataaag

SEQ ID NO: 35 - DNA, 47 bp, Artificial
  ASPE primer
tcttactaatttcaatactcttacttctggagatttatgttctatgg

SEQ ID NO: 36 - DNA, 47 bp, Artificial
  ASPE primer
acacttatctttcaattcaattacttctggagatttatgttctatga

SEQ ID NO: 37 - DNA, 52 bp, Artificial
  ASPE primer
tacacaaacaatctttcacaatttcctatgacccggataacaaggaggaacg

SEQ ID NO: 38 - DNA, 43 bp, Artificial
  ASPE primer
cataactatcaaatatctacactacggataacaaggaggaaca

SEQ ID NO: 39 - DNA, 45 bp, Artificial
  ASPE primer
ctcaattactacatacatttaacagaacgctctatcgcgatttat

SEQ ID NO: 40 - DNA, 45 bp, Artificial
  ASPE primer
tcacttattacttcacattatctagaacgctctatcgcgatttaa

SEQ ID NO: 41 - DNA, 44 bp, Artificial
  ASPE primer
ttcattctcttacattaaacacttatttttggccttcatcacat

SEQ ID NO: 42 - DNA, 44 bp, Artificial
  ASPE primer
acttatttcttcactactatatcaatttttggccttcatcacac

SEQ ID NO: 43 - DNA, 45 bp, Artificial
  ASPE primer
aatctctacaatttctctctaatagcctgtgcaaggaagtattac

SEQ ID NO: 44 - DNA, 47 bp, Artificial
  ASPE primer
tcacatctatttcacaacattattgggcctgtgcaaggaagtattaa

SEQ ID NO: 45 - DNA, 48 bp, Artificial
  ASPE primer
acttacaataactactaatactctacaacctgaacaaatttgatgaag

SEQ ID NO: 46 - DNA, 48 bp, Artificial
  ASPE primer
tactacttctataactcacttaaaacaacctgaacaaatttgatgaat

SEQ ID NO: 47 - DNA, 45 bp, Artificial
  ASPE primer
caaacaaacattcaaatatcaatccacagataaaaacaccacaaa

SEQ ID NO: 48 - DNA, 45 bp, Artificial
  ASPE primer
aatcaacacacaataacattcatacacagataaaaacaccacaag

SEQ ID NO: 49 - DNA, 50 bp, Artificial
  ASPE primer
tttacaaatctaatcacactatacaatgagatggtggtgaatattttccg

SEQ ID NO: 50 - DNA, 50 bp, Artificial
  ASPE primer
tttacaactactaaacacacatttaatgagatggtggtgaatattttcca

SEQ ID NO: 51 - DNA, 42 bp, Artificial
  ASPE primer
ctatctatacatttacaaacattcgccgagtgaccgccatgc

SEQ ID NO: 52 - DNA, 39 bp, Artificial
  ASPE primer
ttcattaaacacaaattctatcacgagtgaccgccatgg

SEQ ID NO: 53 - DNA, 39 bp, Artificial
  ASPE primer
cataatcaatttcaactttctactgagtgaccgccatgt

SEQ ID NO: 54 - DNA, 44 bp, Artificial
  ASPE primer
caaatacataatcttacattcactcccaaatccctgttaaaaac

SEQ ID NO: 55 - DNA, 45 bp, Artificial
  ASPE primer
taaatcaatactcataatctcactccaaatccctgttaaaaaaac

SEQ ID NO: 56 - DNA, 46 bp, Artificial
  ASPE primer
tactcaatacttactatattcatccaaatccctgttaaaaaaaaac

SEQ ID NO: 57 - DNA, 41 bp, Artificial
  ASPE primer
cacttaattcattctaaatctatcagtggatccagcaaccg

SEQ ID NO: 58 - DNA, 42 bp, Artificial
  ASPE primer
taaatacttcattactaatcacaccagtggatccagcaacct

SEQ ID NO: 59 - DNA, 50 bp, Artificial
  ASPE primer
cttaaactctacttacttctaatttcttctagttggcatgctttgatgac

SEQ ID NO: 60 - DNA, 42 bp, Artificial
  ASPE primer
ctctattactactaaatactaatcttggcatgctttgatgaa

SEQ ID NO: 61 - DNA, 48 bp, Artificial
  ASPE primer
tacattcaacactcttaaatcaaaatcataggaaacaccaaagatgat

SEQ ID NO: 62 - DNA, 48 bp, Artificial
  ASPE primer
atctcaattacaataacacacaaatcatcataggaaacaccaaagata

SEQ ID NO: 63 - DNA, 45 bp, Artificial
  ASPE primer
ctttatcaaattctaattctcaacttcatcataggaaacaccaat

SEQ ID NO: 64 - DNA, 46 bp, Artificial
  ASPE primer
acaaatatctaactactatcacaacataggaaacaccaaagatgac

SEQ ID NO: 65 - DNA, 46 bp, Artificial
  ASPE primer
tacaacatctcattaacatatacacatcataggaaacaccaaagac

SEQ ID NO: 66 - DNA, 48 bp, Artificial
  ASPE primer
catacatcttaaatctctttatcttatattcatcataggaaacaccac

SEQ ID NO: 67 - DNA, 50 bp, Artificial
  ASPE primer
tctcaacatataactcatttcttactctaattttctatttttggtaatag

SEQ ID NO: 68 - DNA, 50 bp, Artificial
  ASPE primer
ttaatacaattctctctttctctactctaattttctatttttggtaataa

SEQ ID NO: 69 - DNA, 44 bp, Artificial
  ASPE primer
tctctttaaacacattcaacaataagtgtgattccaccttctcc

SEQ ID NO: 70 - DNA, 46 bp, Artificial
  ASPE primer
ttcaattcaaatcaaacacatcattcagtgtgattccaccttctca

SEQ ID NO: 71 - DNA, 53 bp, Artificial
  ASPE primer
aatatacatctcaaacatttacaccttgctagaccaataattagttattcacc

SEQ ID NO: 72 - DNA, 53 bp, Artificial
  ASPE primer
aatattcactacaatctctacattcttgctagaccaataattagttattcacg

SEQ ID NO: 73 - DNA, 44 bp, Artificial
  ASPE primer
cataaatcttctcattctaacaaagctaaagaaattcttgctcg

SEQ ID NO: 74 - DNA, 44 bp, Artificial
  ASPE primer
ctaactataatcttcatacacatagctaaagaaattcttgctca

SEQ ID NO: 75 - DNA, 48 bp, Artificial
  ASPE primer
cactacacatttatcataacaaatccaataattagttattcaccttgc

SEQ ID NO: 76 - DNA, 48 bp, Artificial
  ASPE primer
actacttattctcaaactctaataccaataattagttattcaccttgt

SEQ ID NO: 77 - DNA, 46 bp, Artificial
  ASPE primer
cttaacatttaacttctataacacaaagaaattcttgctcgttgac

SEQ ID NO: 78 - DNA, 46 bp, Artificial
  ASPE primer
ctttcttaatacattacaacatacaaagaaattcttgctcgttgat

SEQ ID NO: 79 - DNA, 44 bp, Artificial
  ASPE primer
ctacaaacacttaactttatcttttcttgctcgttgacctccac

SEQ ID NO: 80 - DNA, 47 bp, Artificial
  ASPE primer
aataacaactcactatatcataacaattcttgctcgttgacctccat

SEQ ID NO: 81 - DNA, 43 bp, Artificial
  ASPE primer
taatttctttcatctctacataactcttgctcgttgacctcca

SEQ ID NO: 82 - DNA, 43 bp, Artificial
  ASPE primer
atatactttacactttcaacaaactcttgctcgttgacctccc

SEQ ID NO: 83 - DNA, 53 bp, Artificial
  ASPE primer
tttatacatatacacaaactctctattataagtaaggtattcaaagaacatac

SEQ ID NO: 84 - DNA, 53 bp, Artificial
  ASPE primer
ctaaacatacaaatacacatttcaattataagtaaggtattcaaagaacatat

SEQ ID NO: 85 - DNA, 53 bp, Artificial
  ASPE primer
ttaaacaatctactattcaatcacgagcattataagtaaggtattcaaagaac

SEQ ID NO: 86 - DNA, 53 bp, Artificial
  ASPE primer
ctaatctatctttaatcactaaacgagcattataagtaaggtattcaaagaaa

SEQ ID NO: 87 - DNA, 48 bp, Artificial
  ASPE primer
ctaaatcacatacttaacaacaaagtctcctggacagaaacaaaaaaa

SEQ ID NO: 88 - DNA, 48 bp, Artificial
  ASPE primer
tacacaatattcatcataactaacgtctcctggacagaaacaaaaaac

SEQ ID NO: 89 - DNA, 47 bp, Artificial
  ASPE primer
cacatacattattcacaattacatgtctcctggacagaaacaaaaag

SEQ ID NO: 90 - DNA, 47 bp, Artificial
  ASPE primer
atactttacaaacaaataacacaccttacaaatgaatggcatcgaag

SEQ ID NO: 91 - DNA, 47 bp, Artificial
  ASPE primer
ttaacaacttatacaaacacaaaccttacaaatgaatggcatcgaaa

SEQ ID NO: 92 - DNA, 51 bp, Artificial
  ASPE primer
tacttaaacatacaaacttactcacaatctacacaataggacatggaatac

SEQ ID NO: 93 - DNA, 51 bp, Artificial
  ASPE primer
attaaacaactcttaactacacaacaatctacacaataggacatggaatat

SEQ ID NO: 94 - DNA, 50 bp, Artificial
  ASPE primer
ttcttcattaacttctaatcttacacttaacggtacttatttttacatac

SEQ ID NO: 95 - DNA, 50 bp, Artificial
  ASPE primer
ttctttattctcattatcacatcaacttaacggtacttatttttacatat

SEQ ID NO: 96 - DNA, 44 bp, Artificial
  ASPE primer
catcttcatatcaattctcttattctgccaactggttcttgtac

SEQ ID NO: 97 - DNA, 44 bp, Artificial
  ASPE primer
tcaaactctcaattcttacttaatctgccaactggttcttgtag

SEQ ID NO: 98 - DNA, 44 bp, Artificial
  ASPE primer
tctcatctatcatactaattctttctgccaactggttcttgtaa

SEQ ID NO: 99 - DNA, 50 bp, Artificial
  ASPE primer
ttctcttcttcactttatacaaatgatgacaaaaatcatttctattctca

SEQ ID NO: 100 - DNA, 50 bp, Artificial
  ASPE primer
ttcaaacattcaaattactactacgatgacaaaaatcatttctattctct

SEQ ID NO: 101 - DNA, 47 bp, Artificial
  ASPE primer
caaacacaatcaatcttatctattatgtcaatgaacttaaagactcg

SEQ ID NO: 102 - DNA, 47 bp, Artificial
  ASPE primer
attacacacttatcaactaatctaatgtcaatgaacttaaagactca

SEQ ID NO: 103 - DNA, 53 bp, Artificial
  ASPE primer
aactttctctctctattcttatttgccattcttgtatggtttggttgacttgg

SEQ ID NO: 104 - DNA, 53 bp, Artificial
  ASPE primer
aatttcttctctttctttcacaatgccattcttgtatggtttggttgacttgt

SEQ ID NO: 105 - DNA, 46 bp, Artificial
  ASPE primer
tacttctttactacaatttacaactcacacgtgaagaaagatgaca

SEQ ID NO: 106 - DNA, 46 bp, Artificial
  ASPE primer
acaatatacatcacttaaactttctcacacgtgaagaaagatgacg

SEQ ID NO: 107 - DNA, 46 bp, Artificial
  ASPE primer
ataactctcttcacttaaatctattctgttgcagtattaaaatggc

SEQ ID NO: 108 - DNA, 46 bp, Artificial
  ASPE primer
taacttacacttaactatcatctttctgttgcagtattaaaatggt

SEQ ID NO: 109 - DNA, 47 bp, Artificial
  ASPE primer
ctttctcatactttcaactaattttcagggaagagtactttgttatc

SEQ ID NO: 110 - DNA, 47 bp, Artificial
  ASPE primer
cttctattcattaatcattcacaatcagggaagagtactttgttata

SEQ ID NO: 111 - DNA, 43 bp, Artificial
  ASPE primer
attcaatactatctaacacttactcactccaaaggctttcctc

SEQ ID NO: 112 - DNA, 43 bp, Artificial
  ASPE primer
ctattcttacattctaatatctctcactccaaaggctttcctt

SEQ ID NO: 113 - DNA, 42 bp, Artificial
  ASPE primer
acactcatttaacactatttcattttgggaagaactggatca

SEQ ID NO: 114 - DNA, 42 bp, Artificial
  ASPE primer
cttatcataaactctaactttcatttgggaagaactggatcg

SEQ ID NO: 115 - DNA, 49 bp, Artificial
  ASPE primer
tcatcactttctttactttacattgagtactttgttatcagcttttttg

SEQ ID NO: 116 - DNA, 52 bp, Artificial
  ASPE primer
caatttacatttcactttcttatcgaagagtactttgttatcagcttttttt

SEQ ID NO: 117 - DNA, 52 bp, Artificial
  ASPE primer
acatcaaattctttcaatatcttctattttttctggaacatttagaaaaaac

SEQ ID NO: 118 - DNA, 52 bp, Artificial
  ASPE primer
taacataacttcttcattattcactattttttctggaacatttagaaaaaag

(19) United States
(12) Patent Application Publication
(10) Pub. No.: US 2008/0138803 A1
(43) Pub. Date: Jun. 12, 2008

(54) Method of Detecting Cystic Fibrosis Associated Mutations

(76) Inventors: Barbara Galvan-Goldman (nee Galvan), Etobicoke (CA); Connie Lisle, Ancaster (CA)

Correspondence Address: GOODWIN PROCTER LLP;PATENT ADMINISTRATOR, EXCHANGE PLACE, BOSTON, MA, 02109-2881, US

(21) Appl. No.: 11/628,194

(22) PCT Filed: Jun. 1, 2005
(86) PCT No.: PCT/CA05/00829

§ 371 Date: Sep. 10, 2007

Related U.S. Application Data
(60) Provisional application No. 60/575,394, filed on Jun. 1, 2004.

(51) Int. Cl. C12Q 1/68

(52) U.S. Cl. 435/6

(57) ABSTRACT

The present invention describes a method for the simultaneous identification of two or more single base changes, insertions, deletions or translocations in a plurality of target nucleotide sequences that are markers associated with cystic fibrosis. Multiplex detection is accomplished using multiplexed tagged allele specific primer extension (ASPE) and hybridization of such extended primers to a probe, preferably an addressable anti-tagged support.

### BACKGROUND OF THE INVENTION

1. Field of the Invention

The present invention relates to methods and kits for the detection of mutations associated with cystic fibrosis.

2. Description of the Prior Art

Cystic Fibrosis (CF) is the most common autosomal recessive disorder in the Caucasian population, with an incidence of approximately 1 in 3200 live births. The incidence of CF in additional ethnic groups is summarized in Table 1.

TABLE 1
Incidence of CF in Various Ethnic Groups

| Ethnic Group | Incidence of Cystic Fibrosis |
|---|---|
| North American Caucasian | 1 in 3200 |
| Ashkenazi Jewish | 1 in 3300 |
| Hispanic | 1 in 9500 |
| African American | 1 in 15 300 |
| Asian American | 1 in 32 100 |
| Native American (Pueblo) | 1 in 3970 |
| Native American (Zuni) | 1 in 1347 |

CF affects many functions of the body including breathing, digestion and reproduction. Common symptoms of CF include; coughing, wheezing, susceptibility to infections, pneumonia, nasal polyps, digestive problems, inhibited growth, and infertility.

The gene for CF was isolated by positional cloning in 1989 (Rommens, Iannuzzi et al. 1989) and was found to encode a 1480 amino acid transmembrane protein, which was named cystic fibrosis transmembrane conductance regulator (CFTR). The CFTR protein functions as a chloride channel (Bear, Li et al. 1992), and also controls the regulation of other transport pathways (Gabriel, Clarke et al. 1993; Schwiebert, Egan et al. 1995). Mutations in CFTR result in defective chloride ion transport and defective electrolyte transport (Ratjen and Doring 2003).

Over 1200 mutations have been found in the CFTR gene; however many of these mutations have only been found in either single cases, or in a small number of cases. The most common mutation is a three base pair deletion that results in the loss of a phenylalanine at amino acid 508 (ΔF508)—this mutation accounts for 30 to 88 percent of all CF mutations depending on the ethnic group (Gibson, Moskowitz et al. 2001). ΔF508 is the most common mutation in most ethnicities.

The American College of Medical Geneticists (ACMG) and the American College of Obstetricians and Gynecologists (ACOG) has recommended a specific panel of 25 mutations for CF genetic testing, plus reflex testing of four variants. The 25 mutations included in the panel occur at or greater than a frequency of 0.1 percent in the U.S. population as a whole.

Four CFTR variants are recommended for reflex testing: 5T/7T/9T, I506V, I507V, and F508C. Reflex testing is done when positive results are obtained for certain mutations in order to clarify, confirm or expand the positive results.

Table 2 lists the 40 most common mutations which have been associated with CF, as well as the four CFTR variants. The 25 mutations identified by the ACMG and the ACOG are highlighted in bold, and the four CFTR variants are indicated in italics.

TABLE 2
Common Mutations Associated with Cystic Fibrosis

| | | | |
|---|---|---|---|
| ΔF508 | A455E | 3849 + 10kbC→T | 2183AA→G |
| ΔI507 | 1717 − 1G→A | W1282X | 2307insA |
| G542X | R560T | N1303K | Y1092X |
| G85E | R553X | 394delTT | M1101K |
| R117H | G551D | Y122X | S1255X |
| I148T | 1898 + 1G→A | R347H | 3876delA |
| 621 + 1G→T | 2184delA | V520F | 3905insT |
| 711 + 1G→T | 2789 + 5G→A | A559T | 5/7/9T |
| 1078delT | 3120 + 1G→A | S549N | F508C |
| R334W | R1162X | S549R (T→G) | I507V |
| R347P | 3659delC | 1898 + 5G→T | I506V |

The mutations recited in Table 2 are described in further detail in, for example, the articles listed in the reference section below.

Several kits are commercially available for identification of the 25 mutations identified by the ACMG and the ACOG, each of which utilizes a different technology to detect mutations. Such kits have been produced by, for example, Ambion, Celera Diagnostics/Abbott, Roche Diagnostics and Innogenetics, Orchid, Nanogen, Third Wave, and Genzyme.

Multiplex Allele Specific Primer Extension and Solid Support Detection of Mutations

Multiplex allele specific primer extension, and hybridization of extended primers to a solid support is described generally in the prior art. ASPE technology has been generally described in U.S. Pat. No. 4,851,331. The technology is designed to identify the presence or absence of specific polymorphic sites in the genome.

Multiplex ASPE in conjunction with hybridization to a support for mutation detection can be described generally as follows:

1) Amplifying regions of DNA comprising polymorphic loci utilizing a multiplexed, PCR.

2) Allele specific extension of primers wherein the amplified regions of DNA serve as target sequences for the allele specific extension. Extension primers that possess a 3′ terminal nucleotide which form a perfect match with the target sequence are extended to form extension products. Modified nucleotides are incorporated into the extension product, such nucleotides effectively labelling the extension products for detection purposes. Alternatively, an extension primer may instead comprise a 3′ terminal nucleotide which forms a mismatch with the target sequence. In this instance, primer extension does not occur unless the polymerase used for extension possesses exonuclease activity.

3) Hybridizing the extension product to a probe on a solid support, such as a microarray, wherein the probe is complementary to the 5′ end of the extension product.

The extension primers used in a methodology as described above, possess unique sequence tags at their 5′ ends. For example, the sequence tags may allow the extension products to be captured on a solid support.

Variations of the above technology have been described, for example, in U.S. Pat. No. 6,287,778 and PCT Application (WO 00/47766).

ASPE technology may be used to identify numerous types of mutations including, deletions, single nucleotide polymorphisms (SNPs), and insertions.

It is an object of the present invention to provide a convenient and rapid multiplex ASPE/microarray approach for the detection of at least two mutations that have been associated with cystic fibrosis.

### SUMMARY OF THE INVENTION

In one embodiment, the present invention provides a method for detecting the presence or absence of mutations in a sample selected from the group of mutations identified in Table 2, the method comprising the steps of:

Amplifying regions of DNA which may contain the above mentioned mutations using at least two PCR primers pairs selected from the group consisting of SEQ ID NO.: 11 and SEQ ID NO.: 12, SEQ ID NO.: 13 and SEQ ID NO.: 14, SEQ ID NO.: 15 and SEQ ID NO.: 16, SEQ ID NO.: 1 and SEQ ID NO.: 2, SEQ ID NO.: 17 and SEQ ID NO.: 18, SEQ ID NO.: 3 and SEQ ID NO.: 4, SEQ ID NO.: 19 and SEQ ID NO.: 20, SEQ ID NO.: 21 and SEQ ID NO.: 22, SEQ ID NO.: 25 and SEQ ID NO.: 26, SEQ ID NO.: 5 and SEQ ID NO.: 6, SEQ ID NO.: 7 and SEQ ID NO.: 8, SEQ ID NO.: 27 and SEQ ID NO.: 28, SEQ ID NO.: 29 and SEQ ID NO.: 30, SEQ ID NO.: 31 and SEQ ID NO.: 32, SEQ ID NO.: 9 and SEQ ID NO.: 10, and SEQ ID NO.: 23 and SEQ ID NO.: 24.

Hybridizing at least two tagged allele specific extension primers, the allele specific extension primers selected from the group consisting of SEQ ID NO: 33 to SEQ ID NO: 118, to a complementary region of amplified DNA, each tagged allele specific primer having a 3′ portion complementary to a region of the amplified DNA, a 3′ terminal nucleotide complementary to one allele of one of the mutation sites (wild type or mutant) mentioned above, and a 5′ portion complementary to a probe sequence.

Extending tagged ASPE primers, whereby a labelled extension product of the primer is synthesised when the 3′ terminal nucleotide of the primer is complementary to a corresponding nucleotide in the target sequence; no extension product is synthesised when the terminal nucleotide of the primer is not complementary to the corresponding nucleotide in the target sequence.

Hybridizing extension products to a probe and detection of labelled extension products. Detection of a labelled extension product is indicative of the presence of the allele complementary to the 3′-terminal nucleotide of the ASPE primer. In the absence of a labelled extension product, it is determined that the allele corresponding to the 3′ end of the ASPE primer is not present in the sample.

In another embodiment, the present invention provides a kit for use in detecting the presence or absence of at least two mutations identified in Table 2, the kit including at least two tagged allele specific extension primers selected from the group consisting of SEQ ID NO: 33 to SEQ ID NO: 118, and pcr primers pairs selected from the group consisting of SEQ ID NO.: 11 and SEQ ID NO.: 12, SEQ ID NO.: 13 and SEQ IDNO.: 14, SEQ IDNO.: 15 and SEQ ID NO.: 16, SEQ ID NO.: 1 and SEQ ID NO.: 2, SEQ IDNO.: 17 and SEQ IDNO.: 18, SEQ ID NO.: 3 and SEQ IDNO.: 4, SEQ ID NO.: 19 and SEQ ID NO.: 20, SEQ IDNO.: 21 and SEQ ID NO.: 22, SEQ IDNO.: 25 and SEQ ID NO.: 26, SEQ IDNO.: 5 and SEQ ID NO.: 6, SEQ ID NO.: 7 and SEQ IDNO.: 8, SEQ ID NO.: 27 and SEQ ID NO.: 28, SEQ ID NO.: 29 and SEQ IDNO.: 30, SEQ IDNO.: 31 and SEQ ID NO.: 32, SEQ ID NO.: 9 and SEQ IDNO.: 10, and SEQ ID NO.: 23 and SEQ ID NO.: 24.

### BRIEF DESCRIPTION OF THE DRAWINGS

These and other features of the preferred embodiments of the invention will become more apparent in the following detailed description in which reference is made to the appended drawings wherein:

FIG. 1 depicts an example of steps of the present invention.

FIG. 2 is a photograph of a gel presenting results of a genotyping test using the present invention.

### DESCRIPTION OF THE PREFERRED EMBODIMENTS

### Definitions

The following terms used in the present application will be understood to have the meanings defined below.

The term “mutations” as used herein refers to a number of classes of alteration in a nucleotide sequence including but not limited to, deletions, single nucleotide polymorphisms (SNP), and insertions. An example of a deletion is the ΔF508 nucleotide deletion of the CFTR gene associated with CF. An example of an insertion is the 3905insT mutation associated with CF. An example of an SNP is the 3120+1G→A mutation associated with CF.

The terms “oligonucleotide” and “polynucleotide” as used in the present application refer to DNA sequences being of greater than one nucleotide in length. Such sequences may exist in either single or double-stranded form. Examples of oligonucleotides described herein include PCR primers, ASPE primers, and anti-tags.

The term “allele” is used herein to refer to different versions of a nucleotide sequence.

The expression “allele specific primer extension (ASPE)”, as used herein, refers to a mutation detection method utilizing primers which hybridize to a corresponding DNA sequence and which are extended depending on the successful hybridization of the 3′ terminal nucleotide of such primer. Amplified regions of DNA serve as target sequences for ASPE primers. Extension primers that possess a 3′ terminal nucleotide which form a perfect match with the target sequence are extended to form extension products. Modified nucleotides can be incorporated into the extension product, such nucleotides effectively labelling the extension products for detection purposes. Alternatively, an extension primer may instead comprise a 3′ terminal nucleotide which forms a mismatch with the target sequence. In this instance, primer extension does not occur unless the polymerase used for extension inadvertently possesses exonuclease activity.

The term “genotype” refers to the genetic constitution of an organism. More specifically, the term refers to the identity of alleles present in an individual. “Genotyping” of an individual or a DNA sample refers to identifying the nature, in terms of nucleotide base, of the two alleles possessed by an individual at a known polymorphic site.

The term “polymorphism”, as used herein, refers to the coexistence of more than one form of a gene or portion thereof.

The term “PCR”, as used herein, refers to the polymerase chain reaction. PCR is a method of amplifying a DNA base sequence using a heat stable polymerase and a pair of primers, one primer complementary to the (+)-strand at one end of the sequence to be amplified and the other primer complementary to the (−) strand at the other end of the sequence to be amplified. Newly synthesized DNA strands can subsequently serve as templates for the same primer sequences and successive rounds of heat denaturation, primer annealing and strand elongation results in rapid and highly specific amplification of the desired sequence. PCR can be used to detect the existence of a defined sequence in a DNA sample.

The term “primer”, as used herein, refers to a short single-stranded oligonucleotide capable of hybridizing to a complementary sequence in a DNA sample. A primer serves as an initiation point for template dependent DNA synthesis. Deoxyribonucleotides can be joined to a primer by a DNA polymerase. A “primer pair” or “primer set” refers to a set of primers including a 5′upstream primer that hybridizes with the complement of the 5′ end of the DNA sequence to be amplified and a 3′ downstream primer that hybridizes with the 3′ end of the DNA sequence to be amplified. The term “PCR primer” as used herein refers to a primer used for a PCR reaction. The term “ASPE primer” as used herein refers to a primer used for an ASPE reaction.

The term “tag” as used herein refers to an oligonucleotide sequence that is coupled to an ASPE primer. The sequence is generally unique and non-complementary to the human genome while being substantially complementary to a probe sequence. The probe sequence may be, for example, attached to a solid support. Tags serve to bind the ASPE primers to a probe.

The term “tagged ASPE primer” as used herein refers to an ASPE primer that is coupled to a tag.

The term “anti-tag” or “probe” as used herein refers to an oligonucleotide sequence having a sequence complementary to, and capable of hybridizing to, the tag sequence of an ASPE primer. The “anti-tag” may be coupled to a support.

The term “wild type” or “wt” as used herein refers to the normal, or non-mutated, or functional form of a gene.

The term “homozygous wild-type” as used herein refers to an individual possessing two copies of the same allele, such allele characterized as being the normal and functional form of a gene.

The term “heterozygous” or “HET” as used herein refers to an individual possessing two different alleles of the same gene.

The term “homozygous mutant” as used herein refers to an individual possessing two copies of the same allele, such allele characterized as the mutant form of a gene.

The term “mutant” or “mut” as used herein refers to a mutated, or potentially non-functional form of a gene.

The term “call” as used herein refers to the assigned genotype for a particular mutation or variant of the CFTR gene.

The expression “sample failure” as used herein refers to a failure to provide any genotype using a testing method.

### DESCRIPTION OF THE INVENTION

The present invention was developed in response to a need for a rapid, highly specific, and cost-effective method to simultaneously identify multiple genetic risk factors associated with cystic fibrosis. Such identification of risk factors can enhance both treatment and prevention of serious health problems associated with the disease.

The present invention provides a novel, multiplex method of detecting multiple mutations associated with cystic fibrosis. Specifically, the methodology can be used for the detection of the presence or absence of two or more mutations selected from the group consisting of the mutations identified in Table 2. In a preferred embodiment, the present invention provides a method of detecting the presence or absence of all the mutations identified in Table 2.

The positive detection of one or more of the mutations identified in Table 2 may be indicative of an individual having a predisposition for cystic fibrosis.

The present invention is further characterized by a high level of specificity. Such specificity is required in order to ensure that any result generated is a true representation of the genomic target and not simply the result of non-specific interactions occurring between reagents present in reactions. This is especially important for multiplexed DNA-based tests where the numerous sequences present in the reaction mixture, most of which are non-complementary, may interact non-specifically depending on the reaction conditions. The high specificity of the present invention is described by example further below.

The present invention is also characterized by its high level of accuracy when compared to existing methodologies for the detection of mutations associated with cystic fibrosis. An example illustrating the accuracy of the present method is provided further below.

The methodology of the present invention utilizes the combination of multiplex ASPE technology with hybridization of tagged and labelled extension products to probes in order to facilitate detection. Such methodology is suitable for high-throughput clinical genotyping applications.

In one embodiment, the present invention provides a method for detecting the presence or absence of mutations in a sample selected from the group of mutations identified in Table 2, the method comprising the steps of:

Amplifying regions of DNA which may contain the above mentioned mutations.

Hybridizing at least two tagged allele specific extension primers to a complementary region of amplified DNA, each tagged allele specific primer having a 3, portion complementary to a region of the amplified DNA, a 3′ terminal nucleotide complementary to one allele of one of the mutation sites (wild type or mutant) mentioned above, and a 5′ portion complementary to a probe sequence.

Extending tagged ASPE primers, whereby a labelled extension product of the primer is synthesised when the 3′ terminal nucleotide of the primer is complementary to a corresponding nucleotide in the target sequence; no extension product is synthesised when the terminal nucleotide of the primer is not complementary to the corresponding nucleotide in the target sequence.

Hybridizing extension products to a probe and detection of labelled extension products. Detection of a labelled extension product is indicative of the presence of the allele complementary to the 3′-terminal nucleotide of the ASPE primer. In the absence of a labelled extension product, it is determined that the allele corresponding to the 3′ end of the ASPE primer is not present in the sample.

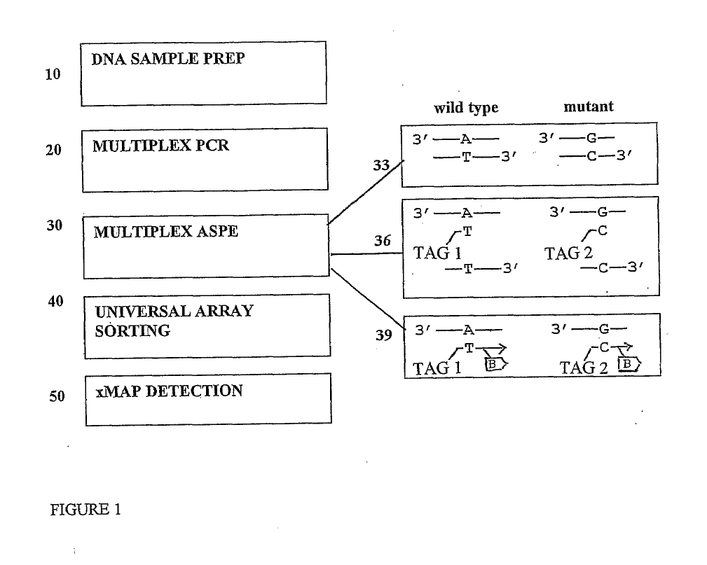

A general overview of one example of the above-mentioned method is presented in FIG. 1. The present invention should not be limited to the example provided in FIG. 1. A DNA sample is first prepared 10 using methods known in the art. Multiplex PCR amplification 20 is conducted in order amplify regions of DNA containing SNP sites that are associated with cystic fibrosis. A multiplex ASPE reaction 30 is then conducted. By example only, 33 illustrates a wild type and a mutant allele of a gene. At step 36 ASPE primers are hybridized to amplified regions of DNA. If the 3′ terminal nucleotide of an ASPE primer is complementary to a corresponding nucleotide in the target sequence, a labelled extension product is formed 39 as will be described further below. The ASPE may be sorted on an addressable universal sorting array 40 wherein the presence of a labelled extension product may be detected using, for example, xMAP detection 50.

### DNA Sample Preparation

Patient samples can be extracted with a variety of methods known in the art to provide nucleic acid (most preferably genomic DNA) for use in the following method.

### Amplification

In a first step at least two regions of DNA containing mutation sites associated with cystic fibrosis are amplified.

In a preferred embodiment of the present invention, PCR amplification of regions containing mutation sites associated with cystic fibrosis is initiated using at least two pairs of PCR primers selected from the group of primer pairs consisting of: SEQ ID NO.: 11 and SEQ ID NO.: 12, SEQ ID NO.: 13 and SEQ ID NO.: 14, SEQ ID NO.: 15 and SEQ ID NO.: 16, SEQ ID NO.: 1 and SEQ ID NO.: 2, SEQ ID NO.: 17 and SEQ ID NO.: 18, SEQ ID NO.: 3 and SEQ ID NO.: 4, SEQ ID NO.: 19 and SEQ ID NO.: 20, SEQ ID NO.: 21 and SEQ ID NO.: 22, SEQ ID NO.: 25 and SEQ ID NO.: 26, SEQ ID NO.: 5 and SEQ ID NO.: 6, SEQ ID NO.: 7 and SEQ ID NO.: 8, SEQ ID NO.: 27 and SEQ ID NO.: 28, SEQ ID NO.: 29 and SEQ ID NO.: 30, SEQ ID NO.: 31 and SEQ ID NO.: 32, SEQ ID NO.: 9 and SEQ ID NO.: 10, and SEQ ID NO.: 23 and SEQ ID NO.: 24.

The relationships of each pair of primers to the mutation sites listed in Table 2 is presented in Table 3.

TABLE 3
Primer Pairs Used to Amplify Regions Containing Cystic Fibrosis
Associated Mutations

| MUTATION | PRIMER PAIR | MUTATION | PRIMER PAIR |
|---|---|---|---|
| ΔF508 | SEQ ID NO.: 11 | R553X | SEQ ID NO.: 13 |
| | SEQ ID NO.: 12 | | SEQ ID NO.: 14 |
| ΔI507 | SEQ ID NO.: 11 | G551D | SEQ ID NO.: 13 |
| | SEQ ID NO.: 12 | | SEQ ID NO.: 14 |
| G542X | SEQ ID NO.: 13 | 1898 + 1G→A | SEQ ID NO.: 15 |
| | SEQ ID NO.: 14 | | SEQ ID NO.: 16 |
| G85E | SEQ ID NO.: 1 | 2184delA | SEQ ID NO.: 17 |
| | SEQ ID NO.: 2 | | SEQ ID NO.: 18 |
| R117H | SEQ ID NO.: 3 | 2789 + 5G→A | SEQ ID NO.: 19 |
| | SEQ ID NO.: 4 | | SEQ ID NO.: 20 |
| I148T | SEQ ID NO.: 3 | 3120 + 1G→A | SEQ ID NO.: 21 |
| | SEQ ID NO.: 4 | | SEQ ID NO.: 22 |
| 621 + 1G→T | SEQ ID NO.: 3 | R1162X | SEQ ID NO.: 25 |
| | SEQ ID NO.: 4 | | SEQ ID NO.: 26 |
| 711 + 1G→T | SEQ ID NO.: 5 | 3659delC | SEQ ID NO.: 25 |
| | SEQ ID NO.: 6 | | SEQ ID NO.: 26 |
| 1078delT | SEQ ID NO.: 7 | 3849 + | SEQ ID NO.: 27 |
| | SEQ ID NO.: 8 | 10kbC→T | SEQ ID NO.: 28 |
| R334W | SEQ ID NO.: 7 | W1282X | SEQ ID NO.: 29 |
| | SEQ ID NO.: 8 | | SEQ ID NO.: 30 |
| R347P | SEQ ID NO.: 7 | N1303K | SEQ ID NO.: 31 |
| | SEQ ID NO.: 8 | | SEQ ID NO.: 32 |
| A455E | SEQ ID NO.: 9 | 394delTT | SEQ ID NO.: 1 |
| | SEQ ID NO.: 10 | | SEQ ID NO.: 2 |
| 1717 − 1G→A | SEQ ID NO.: 13 | Y122X | SEQ ID NO.: 3 |
| | SEQ ID NO.: 14 | | SEQ ID NO.: 4 |
| R560T | SEQ ID NO.: 13 | R347H | SEQ ID NO.: 7 |
| | SEQ ID NO.: 14 | | SEQ ID NO.: 8 |
| A559T | SEQ ID NO.: 13 | S1255X | SEQ ID NO.: 29 |
| | SEQ ID NO.: 14 | | SEQ ID NO.: 30 |
| S549N | SEQ ID NO.: 13 | 3876delA | SEQ ID NO.: 29 |
| | SEQ ID NO.: 14 | | SEQ ID NO.: 30 |
| S549R (T→G) | SEQ ID NO.: 13 | 3905insT | SEQ ID NO.: 29 |
| | SEQ ID NO.: 14 | | SEQ ID NO.: 30 |
| 1898 + 5G→T | SEQ ID NO.: 15 | 5/7/9T | SEQ ID NO.: 9 |
| | SEQ ID NO.: 16 | | SEQ ID NO.: 10 |
| 2183AA→G | SEQ ID NO.: 17 | F508C | SEQ ID NO.: 11 |
| | SEQ ID NO.: 18 | | SEQ ID NO.: 12 |
| 2307insA | SEQ ID NO.: 17 | I507V | SEQ ID NO.: 11 |
| | SEQ ID NO.: 18 | | SEQ ID NO.: 12 |
| Y1092X | SEQ ID NO.: 23 | I506V | SEQ ID NO.: 11 |
| | SEQ ID NO.: 24 | | SEQ ID NO.: 12 |
| M1101K | SEQ ID NO.: 23 | V520F | SEQ ID NO.: 11 |
| | SEQ ID NO.: 24 | | SEQ ID NO.: 12 |

An individual skilled in the art will recognize that alternate PCR primers could be used to amplify the target polymorphic regions, however, in a preferred embodiment the primers listed in Table 3 are selected due to their minimal non-specific interaction with other sequences in the reaction mixture.

### ASPE

The ASPE step of the method of the present invention is conducted using at least two tagged ASPE primers selected from the group of ASPE primers consisting of SEQ ID NO: 33 to SEQ IDNO.: 118.

The ASPE primer set of the present invention has been optimized, as described further below by example, to ensure high specificity and accuracy of diagnostic tests utilizing such allele specific primers.

TABLE 4
ASPE Primers of the Present Invention

| ASPE Primer | SEQ ID NO.: |
|---|---|
| 394delTT-wt | 33 |
| 394delTT-mut | 34 |
| G85E-wt | 35 |
| G85E-mut | 36 |
| R117H-wt | 37 |
| R117H-mut | 38 |
| Y122X-wt | 39 |
| Y122X-mut | 40 |
| I148T-wt | 41 |
| I148T-mut | 42 |
| 621 + 1G->T-wt | 43 |
| 621 + 1G->T-mut | 44 |
| 711 + 1G->T-wt | 45 |
| 711 + 1G->T-mut | 46 |
| 1078delT-wt | 47 |
| 1078delT-mut | 48 |
| R334W-wt | 49 |
| R334W-mut | 50 |
| R347P/H-wt | 51 |
| R347P-mut | 52 |
| R347H-mut | 53 |
| 5T-variant | 54 |
| 7T-variant | 55 |
| 9T-variant | 56 |
| A455E-wt | 57 |
| A455E-mut | 58 |
| V520F-wt | 59 |
| V520F-mut | 60 |
| deltaI507&508-wt | 61 |
| deltaI507-mut(long PITA) | 62 |
| deltaF508-mut | 63 |
| I506V-variant | 64 |
| I507V-variant | 65 |
| F508C-variant | 66 |
| 1717 − 1G->A-wt | 67 |
| 1717 − 1G->A-mut | 68 |
| G542X-wt | 69 |
| G542X-mut | 70 |
| R560T-wt | 71 |
| R560T-mut | 72 |
| R553X-wt | 73 |
| R553X-mut | 74 |
| A559T-wt | 75 |
| A559T-mut | 76 |
| G551D-wt | 77 |
| G551D-mut | 78 |
| S549N-wt | 79 |
| S549N-mut | 80 |
| S549R(T->G)-wt | 81 |
| S549R(T->G)-mut | 82 |
| 1898 + 1G->A-wt | 83 |
| 1898 + 1G->A-mut | 84 |
| 1898 + 5G->T-wt | 85 |
| 1898 + 5G->T-mut | 86 |
| 2184delA-wt | 87 |
| (also wt for | |
| 2183AA->G) | |
| 2184delA-mut | 88 |
| 2183AA->G-mut | 89 |
| 2307insA-wt | 90 |
| 2307insA-mut | 91 |
| 2789 + 5G->A-wt | 92 |
| 2789 + 5G->A-mut | 93 |
| 3120 + 1G>A-wt | 94 |
| 3120 + 1G>A-mut | 95 |
| Y1092X-wt | 96 |
| Y1092X(C->G)- | 97 |
| mut | |
| Y1092X(C->A)- | 98 |
| mut | |
| M1101K-wt | 99 |
| M1101K-mut | 100 |
| R1162X-wt | 101 |
| R1162X-mut | 102 |
| 3659delC-wt | 103 |
| 3659delC-mut | 104 |
| S1255X(19)-wt | 105 |
| S1255X(19)-mut | 106 |
| 3849 + 10kbC->T- | 107 |
| wt | |
| 3849 + 10kbC->T- | 108 |
| mut | |
| S1255X(ex 20)-wt | 109 |
| S1255X(ex 20)- | 110 |
| mut | |
| W1282X-wt | 111 |
| W1282X-mut | 112 |
| 3876delA-wt | 113 |
| 3876delA-mut | 114 |
| 3905insT-wt | 115 |
| 3905insT-mut | 116 |
| N1303K-wt | 117 |
| N1303K-mut | 118 |

Table 4 presents a listing of the ASPE primers used in a preferred embodiment of the present invention. The suffix “wt” represents an ASPE primer used to detect the wild type form of the CFTR gene at a specific mutation site. The suffix “mut” represents an ASPE primer used to detect a mutant form of the CFTR gene at a specific mutation site. Bases 1 to 24 of each of SEQ ID NO.: 33 to SEQ ID NO: 118 are the 5′ portions of the ASPE primers that are complementary to specific probe sequences. Although the specific sequences listed in table 4 are preferred, in alternate embodiments of the present invention, it is possible to combine different 5′ portions of the sequences in Table 4 (bases 1 to 24 of SEQ ID NOs: 33 to 118) with different 3′ end hybridizing portions of the sequences in Table 4 (bases 25 and up of SEQ ID NOs: 33 to 118).

The 3′ end hybridizing portion of the extension primer is hybridized to the amplified material. Where the 3′ terminal nucleotide of an ASPE primer is complementary to the polymorphic site, primer extension is carried out using a modified nucleotide. Where the 3′ terminal nucleotide of the ASPE primer is not complementary to the polymorphic region, no primer extension occurs.

In one embodiment, labelling of the extension products is accomplished through the incorporation of biotinylated nucleotides into the extension product which may be identified using fluorescent (Streptavidin-Phycoerythrin) or chemiluminescent (Streptavidin-Horseradish Peroxidase) reactions. However, an individual skilled in the art will recognize that other labelling techniques may be utilized. Examples of labels useful for detection include but are not limited to radiolabels, fluorescent labels (e.g fluorescein and rhodamine), nuclear magnetic resonance active labels, positron emitting isotopes detectable by a positron emission tomography (“PET”) scanner, and chemiluminescers such as luciferin, and enzymatic markers such as peroxidase or phosphatase.

Each ASPE primer used in the methodology as described above, possess a unique sequence tag at their 5′ ends. The sequence tags allow extension products to be detected with a high degree of specificity, for example, through capture on a solid support in order to facilitate detection.

### Detection

The tagged 5′ portions of the allele specific primers of the present invention are complementary to probe sequences. Upon hybridization of the allele specific primers to a corresponding probe sequence the presence of extension products can be detected.

In a preferred embodiment, probes used in the methodology of the present invention are coupled to a solid support, for example a ‘universal’ bead-based microarray.

Examples of supports that can be used in the present invention include, but are not limited to, bead based microarrays and 2D glass microarrays. The preparation, use, and analysis of microarrays are well known to persons skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), PCT Application WO 95/251116; Shalon, D. et al. (I 995) PCT application WO 95/35505; Heller, R. A. et al. (1997) Proc. Natl. Acad. Sci. 94:2150-2155; and Heller, M. J. et al. (1997) U.S. Pat. No. 5,605,662.). Detection can be achieved through arrays using, for example, chemiluminescence or fluorescence technology for identifying the presence of the SNPs.

Universal arrays function as sorting tools indirectly detecting the target of interest and are designed to be isothermal and minimally cross-hybridizing as a set. Examples of nicroarrays which can be used in the present invention include, but should not be limited to, Luminex's® bead based microarray systems, and Metrigenix's™ Flow Thru chip technology.

In one embodiment, for example, Luminex's 100 xMAP™ fluorescence based solid support microarray system is utilized. Anti-tag sequences complementary to the tag regions of the ASPE primers/extension products, described above, are coupled to the surface of internally fluorochrome-color-coded microspheres. An array of anti-tag microspheres is produced, each set of microspheres having its own characteristic spectral address. The mixture of tagged, extended, biotinylated ASPE primers is combined with the array of anti tagged microspheres and is allowed to hybridize under stringent conditions.

In a reaction mixture, a fluorescent reporter molecule (e.g. streptavidin-phycoerythrin) is used to detect labelled extension products which are synthesized when the terminal nucleotide of an ASPE primer is complementary to a corresponding nucleotide in the target sequence.

The reaction mixture, comprising microspheres, extension products etc. is injected into a reading instrument, for example Lurninex's 100 xMAP™, which uses microfluidics to align the microspheres in single file. Lasers are used to illuminate the colors both internal to the microspheres, and attached to the surface in the form of extension products hybridized to anti-tag sequences. The Luminex 100 xMAP™, interprets the signal received and identifies the presence of wild type and/or mutant alleles. The presence of the mutant allele of any one or more of the 44 mutations presented in Table 2 may be indicative of cystic fibrosis, or a pre-disposition to cystic fibrosis. Software can be provided which is designed to analyze data associated with the specific extension products and anti-tagged microspheres of the present invention.

In another embodiment, the Metrigenix Flow-Thru three dimensional microchannel biochip (Cheek, B. J., Steel A. B., Torres, M. P., Yu, Y., and Yang H. Anal. Chem. 2001, 73, 5777-5783) is utilized for genotyping as known in the art. In this embodiment, each set of microchannels represents a different universal anti-tag population. Anti-tag sequences corresponding to the tag regions of the ASPE primers/extension products, described above, are attached to the inner surface of multiple microchannels comprising a cell. Multiple cells make up a chip. The reaction mixture, including biotinylated extension products flows through the cells in the presence of a chemiluminescent reporter substrate such as streptavidin-horseradish peroxidase. Microarray chips can be imaged using technology known in the art, such as an ORCA-ER CCD (Hamamatsu Photonics K. K., Hamamatsu City, Japan), and imaging software, in order to identify the genotype of an individual.

### Kits

In an additional embodiment, the present invention provides kits for the multiplex detection of mutations associated with cystic fibrosis.

A kit that can be used for detection of the mutations of interest may contain the following components including: a PCR primer mix for amplifying regions containing mutation sites of interest (optionally including dNTPs), an ASPE primer mix for generation of labelled extension products (optionally including dNTPs) and a solid support, such as microarray beads, the beads having anti-tags complementary to the tagged regions of the ASPE primers. In addition, an individual skilled in the art would recognize other components which could be included in such kits including, for example, buffers and polymerases.

Kits of the present invention may include PCR primer pairs, ASPE primers, and tagged supports for all the mutations to be detected, or may be customized to best suit the needs of an individual end user. For example, if an end user wishes to detect only 25 of the mutations associated with cystic fibrosis, a kit can be customized to include only the PCR primer pairs, ASPE primers, and support required for the detection of the desired mutations. As such, the end 30 user of the product can design a kit to match their specific requirements. In addition, the end user can also control the tests to be conducted at the software level when using, for example, a universal bead based-microarray for detection. For example, software can be provided with a kit, such software reading only the beads for the desired mutations or by reporting only the results from the desired mutation data. Similar control of data reporting by software can be obtained when the assay is performed on alternate platforms.

An individual skilled in the art will recognize that although the present method has been described in relation to 44 specific cystic fibrosis associated mutations, PCR primers and ASPE primers used to detect additional mutations could be included in the above method and kits.

All publications, patents and patent applications are herein incorporated by reference in their entirety to the same extent as if each individual publication, patent or patent application was specifically and individually indicated to be incorporated by reference in its entirety

The examples presented below are provided to illustrate the present invention and are not meant to limit the scope of the invention as will be apparent to persons skilled in the art.

### Example #1

### ASPE/Microarray Detection of CFTR Mutations

### MATERIALS and METHODS

1) Oligonucleotides

All oligonucleotides were synthesized by Integrated DNA Technologies (Coralville, Iowa). PCR primers were unmodified and were purified by standard desalting procedures. Universal anti-tags (probes) were 3′-C7 amino-modified for coupling to carboxylated microspheres. All anti-tags were reverse phase HPLC-purified. Chimeric ASPE primers which consisted of a 24mer universal tag sequence 5′ to the allele-specific sequence were also unmodified but were purified by polyacrylamide gel electrophoresis. Following reconstitution, exact oligo concentrations were determined spectrophotometrically using extinction coefficients provided by the supplier. Reconstituted oligos were scanned between 200 and 800 mm and absorbance was measured at 260 nm to calculate oligo concentration.

2) Reagents

Platinum Taq, Platinum Tsp, individual dNTPs and biotin-dCTP were purchased from Invitrogen Corporation (Carlsbad, Calif.). Shrimp alkaline phosphatase and exonuclease I were purchased from USB Corporation (Cleveland, Ohio). Carboxylated fluorescent microspheres were provided by Luminex Corporation (Austin, Tex.). The EDC cross-linker (1-ethyl-3-(3-dimethylaminopropyl)carbodiimide hydrochloride) was purchased from Pierce (Rockford, Ill.). OmniPur reagents including MES (2-(N-morpholino)ethane sulfonic acid), 10% SDS, NaCl, Tris, Triton X-100, Tween-20 and TE buffer were purchased from EM Science (Darmstadt, Germany). The streptavidin-conjugated phycoerythrin was obtained from Molecular Probes Inc. (Eugene, Oreg.).

3) Genotyping

a) MULTIPLEX PCR (16-plex): Multiplex PCR was carried out using 25 ng genomic DNA in a 25 uL final volume. A ‘no target’ PCR negative control was included with each assay run. The reaction consisted of 30 mmol/L Tris-HCl, pH 8.4, 75 mmol/L KCl, 2 mmol/L MgCl₂, 200 umol/L each dNTP, 5 units Platinum Taq, with primers ranging from 0.15 to 0.6 umol/L. Samples were cycled in an MJ Research PTC-200 thermocycler (keno, NV) with cycling parameters set at 95° C. for 5 minutes followed by 30 cycles at 95° C. for 30 seconds, 58° C. for 30 seconds and 72° C. for 30 seconds. Samples were then held at 72° C. for 5 minutes and kept at 4° C. until use.

b) ALLELE-SPECIFIC PRIMER EXTENSION: Prior to the ASPE reaction, each PCR reaction was treated with shrimp alkaline phosphatase (SAP) to inactivate any remaining nucleotides (particularly dCTP) so that biotin-dCTP could be efficiently incorporated during the primer extension reaction. Each PCR reaction was also treated with exonuclease I (EXO) to degrade remaining PCR primers in order to avoid any interference with the tagged ASPE primers and the extension reaction itself. To each 25 uL PCR reaction, 2.5 uL SAP (=2.5 units) and 1.0 uL EXO (=10 units) were added directly. Samples were then incubated at 37° C. for 30 minutes followed by a 15 minute incubation at 99° C. to inactivate the enzymes. Samples were then added directly to the ASPE reaction.

Multiplex ASPE was carried out using 5 uL of treated PCR product in a final volume of 20 uL. Each reaction consisted of 20 mmol/L Tris-HCl pH 8.4, 50 mmol/L KCl, 1.25 mmol/L MgCl2, 5 umol/L biotin-dCTP, 5 umol/L each of dATP, dGTP and dTTP, 4.5 units Platinum Tsp and 10 nmol/L ASPE primer pool (ie. each ASPE primer present at 200 fmol/reaction). The ASPE reactions were incubated at 96° C. for 2 minutes and then subjected to 40 cycles at 94° C. for 30 seconds, 52° C. for 30 seconds and 74° C. for 60 seconds. Reactions were then held at 4° C. until use.

c) BEAD COUPLING: Amino-modified anti-tag sequences were coupled to carboxylated microspheres following Luminex's one-step carbodiimide coupling procedure. Briefly, 5×10⁶ microspheres were combined with 1 mmol NH₂-oligo in a final volume of 50 uL 0.1 mol/L MES, pH 4.5. A 10 mg/mL EDC working solution was prepared just prior to use and 2.5 uL was added to the bead mixture and incubated for 30 minutes. A second 2.5 uL aliquot of freshly prepared EDC was added followed by an additional 30 minute incubation. Following washes in 0.02% (v/v) Tween-20 and 0.1% (w/v) SDS, the anti-tag coupled beads were resuspended in 100 uL TE buffer (10 mmol/L Tris, pH 8.0, 1 mmol/L EDTA). Bead concentrations were determined using a Beckman Coulter Z2 Particle Count and Size Analyzer (Coulter Corp, Miami Fla.).

d) UNIVERSAL ARRAY HYBRIDIZATION: Each hybridization reaction was carried out using approximately 2500 beads of each of the 86 anti-tag bearing bead populations. The beads were combined in hybridization buffer (0.22 mol/L NaCl, 0.11 mol/L Tris, pH 8.0 and 0.088% (v/v) Triton X-100) and 45 uL of the mix were added to each well of an MJ Research 96-well plate (Reno, Nev.). A 5 uL aliquot of each ASPE reaction was then added directly to each well. The samples were then heated to 96° C. for 2 minutes in an MJ Research PTC-200 followed by a one hour incubation at 37° C. Following this incubation, samples were filtered through a 1.2 um Durapore Membrane (Millipore Corp, Bedford, Mass.) and washed once using wash buffer (0.2 mol/L NaCl, 0.1 mol/L Tris, pH 8.0 and 0.08% (v/v) Triton X-100). The beads were then resuspended in 150 uL reporter solution (1 ug/mL streptavidin-conjugated phycoerythrin in wash buffer) and incubated for 15 minutes at room temperature. The reactions were read on the Luminex xMAP. Acquisition parameters were set to measure 100 events per bead population and a 100 uL sample volume. A gate setting was established prior to running the samples and maintained throughout the course of the study.

RESULTS

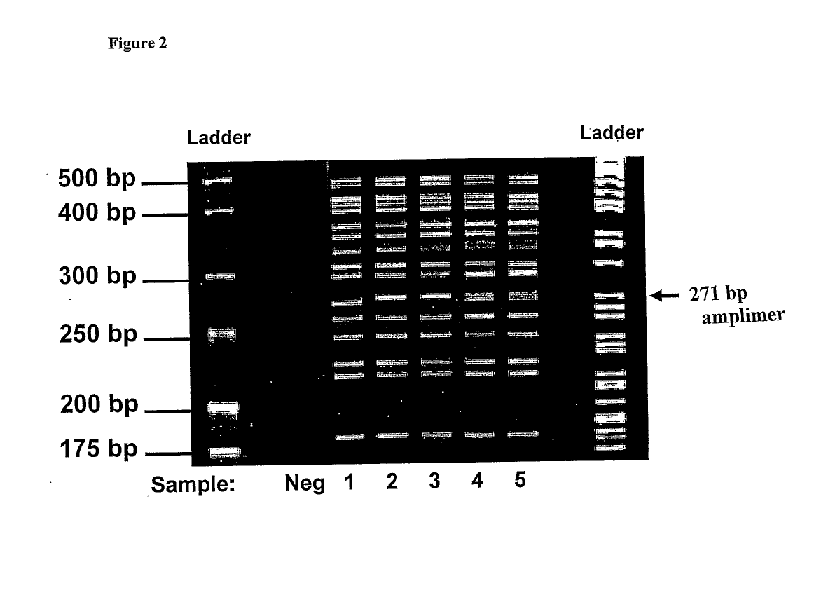

For optimal PCR, buffer composition, cycling parameters, annealing temperature, genomic DNA input as well as primer concentrations for each mutation were examined. PCR products generated under the final optimized conditions were analyzed by gel electrophoresis using the Helixx SuperGel350 system (Scarborough, ON) which is capable of resolving 2-5 basepair differences within products below 500 bp. A gel image of 5 patient samples amplified under optimal conditions is provided in FIG. 2 and clearly demonstrates that the multiplex PCR reaction of the present invention was highly specific for the desired amplimers. The migration and number of bands seen at 271 bp (size of the amplimer containing the ΔF508 mutation) corresponds to the genotype of the sample for the ΔF508 mutation.

### Example #2

This example illustrates both the accuracy and the specificity of the present invention. Accuracy, is a measure of concordance of the resultant genotyping calls on the 44 mutations/variants determined by the method of the present invention (from hereon in this example referred to as the CFTR 40+4 genotyping assay) to the genotyping calls from reference methods.

The reference methods used were (1) DNA sequencing employed by Genaissance Pharmaceuticals and (2) the Applied Biosystems, Inc. Cystic Fibrosis (ABI-CF) System.

The present invention was used to analyze 139 genomic DNA samples. All 139 genomic DNA samples analyzed with the CFTR 40+4 genotyping assay provided calls for all 44 mutations and variants detected by the CFTR 40+4 genotyping assay. Thus, >95% of the genomic DNA samples tested yielded genotyping calls over all 44 mutations and variants tested for by the CFTR 40+4 genotyping assay.

In this example, there were initially a maximum of 6116 genotypic calls possible (139 samples, each genotyped for 44 mutations/variants) by each of either the CFTR 40+4 genotyping assay or by the reference methods. This total number of comparable calls was reduced by 37 calls due to the inability of the DNA sequencing to genotype 37 separate mutations/variants of 24 individual DNA samples. A further two genotyping calls were removed from analysis because of an allele that was not detectable by the CFTR 40+4 genotyping assay. Thus, there were ultimately a total of 6077 possible calls (i.e.: 6116-37-2) that could be made by, and compared between the CFTR 40+4 genotyping assay and the reference methods.

Upon initial comparison of the calls obtained from the CFTR 40+4 genotyping assay and the corresponding calls obtained by the reference methods, eight discordant calls were identified, giving an initial overall percent concordance of 99.87% for the method of the present invention.

The DNA samples with discordant genotyping calls were reanalyzed by DNA sequencing. Upon reanalysis of the eight discordant calls made by the CFTR 40+4 genotyping assay, all mutations/variants were resolved and found to be concordant to the corresponding calls made by DNA sequencing. Therefore, after resolving the discordances the overall percent concordance of the CFTR 40+4 genotyping assay to the reference methods was 100%.

Finally, each of the 44 mutations and variants detected by the CFTR 40+4 genotyping assay initially yielded overall percent concordances of >95%, when compared to the results obtained by the reference methods.

### INTRODUCTION AND BACKGROUND INFORMATION

An overview of the protocol used for this example is outlined below.

For each sample, 25 ng of genomic DNA was amplified in a single multiplex (16-plex) polymerase chain reaction (PCR). The amplimer sizes ranged from 179 bp to 465 bp. To enable efficient incorporation of biotin-dCTP during the multiplex Allele Specific Primer Extension (ASPE) reaction, each PCR product was treated with Shrimp Alkaline Phosphatase (SAP) to inactivate any remaining nucleotides (especially dCTP), and with Exonuclease I (EXO) to degrade any primers left over from the PCR. A 5 μL aliquot of the treated PCR product was used in the ASPE reaction containing 86 universally-tagged primers. The ASPE products are then sorted by hybridization to the universal array (Bead Mix) in the presence of a hybridization buffer, and then incubated with Streptavidin, R-Phycoerythrin conjugate (reporter solution). Samples were read on the Luminex® 100 xMAP™ instrument and a signal was generated for each of the 40 mutations and 4 variants (and/or their corresponding wild-type alleles). These fluorescence values were then analyzed to determine whether for each mutation or variant the samples were wild-type, heterozygous or that at least one mutant allele is present.

Overview of Testing Procedures

One hundred and thirty nine (139) genomic DNA samples were obtained as solutions. These DNA samples were analyzed with the method of the present invention. The DNA samples were previously characterized for 30 of the 44 mutations/variants utilizing the Applied Biosystems, Inc. Cystic Fibrosis System. The remaining 14 mutations and variants not detected by the ABI-CF System were genotyped by DNA sequencing (the other reference method) which was performed by Genaissance Pharmaceuticals. Table 5 indicates the different methodologies and the mutations and/or variants that were genotyped by each method.

TABLE 5
Methods employed to genotype the 44 mutations and variants of the CFTR
that are detected by the method of the present invention.

| | | Mutations/Variants | | |
|---|---|---|---|---|
| Mutations/Variants | | genotyped by DNA | | |
| genotyped via the | | sequencing | Mutations/Variants | |
| CFTR40 + 4 genotyping | | (Genaissance | genotyped via the ABI | |
| assay | | Pharmaceuticals)^{a} | CF System (Dr. Ray) | |
| G85E | R117H | N/A | G85E | R117H |
| Y122X | R334W | | Y122X | R334W |
| R347H | R347P | | R347H | R347P |
| A455E | ΔI507 | | A455E | ΔI507 |
| ΔF508 | V520F | | ΔF508 | V520F |
| G542X | S549N | | G542X | S549N |
| S549R | G551D | | S549R | G551D |
| R553X | R560T | | R553X | R560T |
| 621 + 1G > T | 711 + 1G > T | | 621 + 1G > T | 711 + 1G > T |
| 1078delT | R1162X | | 1078delT | R1162X |
| W1282X | N1303K | | W1282X | N1303K |
| 1717 − 1G > A | 1898 + 1G > A | | 1717 − 1G > A | 1898 + 1G > A |
| 2183AA > G | 2789 + 5G > A | | 2183AA > G | 2789 + 5G > A |
| 3659delC | 3849 + 10kbC > T | | 3659delC | 3849 + 10kbC > T |
| 3905insT | F508C | | 3905insT | F508C |

| | | | | |
|---|---|---|---|---|
| 394delTT | I148T | 394delTT | I148T | N/A |
| 5T/7T/9T | I506V | 5T/7T/9T | I506V | |
| I507V | A559T | I507V | A559T | |
| 1898 + 5G > T | 2184delA | 1898 + 5G > T | 2184delA | |
| 2307insA | 3120 + 1G > A | 2307insA | 3120 + 1G > A | |
| M1101K | | M1101K | | |
| Y1092X (C > G or C > A) | | Y1092X (C > G or C > A) | | |
| S1255X (exon 19 and 20) | | S1255X (exon 19 and 20) | | |
| 3876delA | | 3876delA | | |

^{a}Some of the mutations and variants, that were not detected by the ABI-CF system, although not initially sequenced may be and in some cases were analyzed by DNA sequencing since these mutations and variants were located within the DNA sequence of exons that were shipped to Genaissance Pharmaceuticals specifically for DNA sequencing.

The resulting genotype calls for the 44 mutations and variants obtained from the CFTR 40+4 genotyping assay were then compared to the corresponding calls obtained by the reference methods (DNA sequencing and the ABI-CF System). These comparisons were used to determine the degree of concordance between the calls made by the CFTR 40+4 genotyping assay and the corresponding calls made by the reference methods. This accuracy measure of the CFTR 40+4 genotyping assay was determined for the calling of each particular variant and for the CFTR 40+4 genotyping assay as a whole.

Calculation of Accuracy

The concordance of the genotypic calls obtained by the CFTR 40+4 genotyping assay to the reference methods was determined for each of the CFTR 40+4 mutation and variants per DNA sample. The overall percent concordance was then determined for each mutation and variant over the full genomic DNA sample set. Finally, the overall percent concordance of the method of the present invention was determined from the percentage of genotyping calls determined by the CFTR 40+4 genotyping assay that matched those determined by the reference methods. The formulas used in the calculation of percent concordance are presented in Appendix 1.

Results that yielded a: (1) variant failure, or (2) sample failure were not included in the accuracy calculations since these events do not report a call, but instead indicated a failure of the method or assay used to genotype the mutation or variant.

Acceptance Criteria

The acceptance criteria for the method of the present invention were as follows:
No more than 5% of the samples were allowed to fail (i.e.: not providing any genotype at all).
Of the samples which do not fail, each variant must have an overall % concordance of at least 95% (i.e.: C_{Overall}(m)≧95% for all 44 mutations and variants).
A minimum overall assay % concordance, for the method of the present invention, of at least 98% must be achieved (i.e.: C_{TagIt}≧98%).

Results

Results from the ABI-CF. System Reference Method

TABLE 6
Genotyping calls obtained by the reference methods and the CFTR 40 + 4 genotyping
assay for the mutations/variants and T-tract of DNA Samples 1-18

| | | | | | | | | | | T-Tract variant | |
|---|---|---|---|---|---|---|---|---|---|---|---|
| Tm | | | | | | Mutation/Variant 2 | | | | Reference | Tag-It |
| Sample | HSC | Mutation/Variant 1 | | | | | | Tag-It CFTR | | method | CFTR |
| #^{a} | Sample # | Reference method call | | Tag-It CFTR 40 + 4 call | | Reference method call | | 40 + 4 call | | call | 40 + 4 call |
| 1 | 10821 | 3659delC | HET | 3659delC | HET | ΔF508 | HET | ΔF508 | HET | 7T/9T | 7T/9T D |
| 2 | 10839 | 3659delC | HET | 3659delC | HET | | | | | 7T/9T | 7T/9T D |
| 3 | 10988 | R1162X | HET | R1162X | HET | ΔF508 | HET | ΔF508 | HET | 7T/9T | 7T/9T D |
| 4 | 11155 | N1303K | HET | N1303K | HET | ΔF508 | HET | ΔF508 | HET | 9T | 9T D |
| 5 | 11160 | N1303K | HET | N1303K | HET | | | | | 7T/9T | 7T/9T D |
| 6 | 11332 | N1303K | HET | N1303K | HET | ΔF508 | HET | ΔF508 | HET | 9T | 9T D |
| 7 | 11379 | R560T | HET | R560T | HET | | | | | 7T | 7T D |
| 8 | 11380 | R560T | HET | R560T | HET | | | | | 5T/7T | 5T/7T D |
| 9 | 11688 | 3849 + 10kbC > T | HET | 3849 + 10kbC > T | HET | | | | | 7T/9T | 7T/9T D |
| 10 | 11689 | 3849 + 10kbC > T | HET | 3849 + 10kbC > T | HET | ΔF508 | HET | ΔF508 | HET | 9T | 9T D |
| 11 | 11839 | R1162X | HET | R1162X | HET | ΔF508 | HET | ΔF508 | HET | 7T/9T | 7T/9T D |
| 12 | 11843 | G542X | HET | G542X | HET | | | | | 7T/9T | 7T/9T D |
| 13 | 11858 | R560T | HET | R560T | HET | | | | | 7T/9T | 7T/9T D |
| 14 | 12261 | R560T | HET | R560T | HET | ΔF508 or ΔI507 | HET | ΔF508 | HET | 7T/9T | 7T/9T D |
| 15^{b} | 12394 | R560T | HET/WT | R560T WT | WT | ΔF508 or ΔI507 | HET | ΔF508 | HET | 7T/9T | 7T/9T D |
| 16 | 12846 | R117H | HET | R117H | HET | ΔF508 | HET | ΔF508 | HET | 7T/9T | 7T/9T D |
| 17 | 12897 | R560T | HET | R560T | HET | | | | | 7T | 7T D |
| 18 | 13245 | 3849 + 10kbC > T | HET | 3849 + 10kbC > T | HET | | | | | 7T | 7T D |

^{a}DNA samples were assigned new consecutive numbers at Tm, starting from Sample 1 (i.e.: HSC sample 10821).
^{b}For the R506T mutation, DNA sample 15 was genotyped as HET by the ABI-CF System and WT by both DNA sequencing and the CFTR 40 + 4 genotyping assay. The ABI-CF System detected a third mutation (R560K) and genotyped it as a HET, this mutation is not detected by the CFTR 40 + 4 genotyping kit.

TABLE 7
Genotyping calls obtained by the reference methods and the CFTR 40 + 4 genotyping
assay for the mutations/variants and T-tract of DNA Samples 19-36

| | | | | | | | | | | T-Tract variant | |
|---|---|---|---|---|---|---|---|---|---|---|---|
| Tm | HSC | | | | | | | | | Reference | Tag-It |
| Sample | Sample | Mutation/Variant 1 | | | | Mutation/Variant 2 | | | | method | CFTR |
| # | # | Reference method call | | Tag-It CFTR 40 + 4 call | | Reference method call | | Tag-It CFTR 40 + 4 call | | call | 40 + 4 call |
| 19 | 13246 | 3849 + 10kbC > T | HET | 3849 + 10kbC > T | HET | | | | | 7T | 7T D |
| 20 | 13463 | G542X | HET | G542X | HET | ΔF508 | HET | ΔF508 | HET | 9T | 9T D |
| 21 | 13719 | W1282X | HET | W1282X | HET | | | | | 7T | 7T D |
| 22 | 13763 | W1282X | HET | W1282X | HET | | | | | 7T | 7T D |
| 23^{a} | 13893 | | | | | | | | | | |
| 24^{a} | 13894 | | | | | | | | | | |
| 25 | 13901 | W1282X | HET | W1282X | HET | | | | | 7T | 7T D |
| 26 | 14055 | W1282X | HET | W1282X | HET | | | | | 7T | 7T D |
| 27 | 14341 | W1282X | HET | W1282X | HET | | | | | 7T/9T | 7T/9T D |
| 28 | 14364 | 3849 + 10kbC > T | HET | 3849 + 10kbC > T | HET | | | | | 7T | 7T D |
| 29 | 14705 | W1282X | HET | W1282X | HET | | | | | 7T | 7T D |
| 30 | 15054 | R117H | HET | R117H | HET | ΔF508 | HET | ΔF508 | HET | 7T/9T | 7T/9T D |
| 31 | 15102 | W1282X | HET | W1282X | HET | | | | | 7T | 7T D |
| 32 | 15249 | W1282X | HET | W1282X | HET | | | | | 7T | 7T D |
| 33 | 15265 | W1282X | HET | W1282X | HET | | | | | 7T | 7T D |
| 34 | 15574 | 2789 + 5G > A | HET | 2789 + 5G > A | HET | ΔF508 | HET | ΔF508 | HET | 7T/9T | 7T/9T D |
| 35 | 15866 | R334W | HET | R334W | HET | | | | | 7T | 7T D |
| 36^{b} | 15867 | R334W | HET | R334W | HET | 1898 + 1G > A | ND/ | 1898 + 1G > A | HET | 7T | 7T D |
| | | | | | | | HET | | | | |

^{a}DNA samples 23 and 24 were not tested with the CFTR 40 + 4 genotyping assay.
^{b}For the 1898 + 1G > A mutation, DNA sample 36 was genotyped as WT by the ABI-CF System and HET by both DNA sequencing and the CFTR 40 + 4 genotyping assay.

TABLE 8
Genotyping calls obtained by the reference methods and the CFTR 40 + 4 genotyping
assay for the mutations/variants and T-tract of DNA Samples 37-54

| | | | | | | | | | | T-Tract variant | |
|---|---|---|---|---|---|---|---|---|---|---|---|
| Tm | | | | | | | | | | Reference | Tag-It |
| Sample | HSC | Mutation/Variant 1 | | | | Mutation/Variant 2 | | | | method | CFTR |
| # | Sample # | Reference method call | | Tag-It CFTR 40 + 4 call | | Reference method call | | Tag-It CFTR 40 + 4 call | | call | 40 + 4 call |
| 37^{a} | 15869 | 1898 + 1G > A | ND/HET | 1898 + 1G > A | HET | | | | | 7T | 7T D |
| 38^{b} | 16143 | G542X | HET | G542X | HET | ΔF508 | HET/WT | ΔF508 | WT | 7T/9T | 7T/9T D |
| 39 | 16299 | R334W | HET | R334W | HET | ΔF508 | HET | ΔF508 | HET | 7T/9T | 7T/9T D |
| 40 | 16331 | R334W | MUT | R334W | Mu D | | | | | 7T | 7T D |
| 41 | 17661 | 621 + 1G > T | HET | 621 + 1G > T | HET | ΔF508 | HET | ΔF508 | HET | 9T | 9T D |
| 42 | 17755 | 3659delC | HET | 3659delC | HET | ΔF508 | HET | ΔF508 | HET | 7T/9T | 7T/9T D |
| 43 | 18359 | G85E | HET | G85E | HET | ΔF508 | HET | ΔF508 | HET | 7T/9T | 7T/9T D |
| 44 | 18507 | R1162X | HET | R1162X | HET | ΔF508 | HET | ΔF508 | HET | 7T/9T | 7T/9T D |
| 45 | 18508 | R1162X | HET | R1162X | HET | | | | | 7T | 7T D |
| 46 | 18605 | R334W | MUT | R334W | Mu D | | | | | 7T | 7T D |
| 47 | 19378 | R334W | HET | R334W | HET | | | | | 7T | 7T D |
| 48 | 19568 | 1078delT | HET | 1078delT | HET | ΔF508 | HET | ΔF508 | HET | 7T/9T | 7T/9T D |
| 49 | 20883 | 711 + 1G > T | HET | 711 + 1G > T | HET | 3905insT | HET | 3905insT | HET | 7T | 7T D |
| 50 | 20934 | G542X | HET | G542X | HET | | | | | 7T/9T | 7T/9T D |
| 51 | 22462 | 2789 + 5G > A | HET | 2789 + 5G > A | HET | | | | | 7T | 7T D |
| 52 | 22518 | G542X | HET | G542X | HET | ΔF508 | HET | ΔF508 | HET | 9T | 9T D |
| 53 | 22963 | ΔI507 | HET | ΔI507 | HET | | | | | 7T | 7T D |
| 54 | 23235 | G542X | HET | G542X | HET | | | | | 9T | 9T D |

^{a}For the 1898 + 1G > A mutation, DNA sample 37 was genotyped as WT by the ABI-CF System and HET by both DNA sequencing and the CFTR 40 + 4 genotyping assay.
^{b}For the 1898 + 1G > A mutation, DNA sample 38 was genotyped as HET by the ABI-CF System and WT by both DNA sequencing and the CFTR 40 + 4 genotyping assay.

TABLE 9
Genotyping calls obtained by the reference methods and the CFTR 40 + 4 genotyping
assay for the mutations/variants and T-tract of DNA Samples 55-72

| | | | | | | | | | | T-Tract variant | |
|---|---|---|---|---|---|---|---|---|---|---|---|
| Tm | | | | | | | | | | Reference | Tag-It |
| Sample | HSC | Mutation/Variant 1 | | | | Mutation/Variant 2 | | | | method | CFTR |
| # | Sample # | Reference method call | | Tag-It CFTR 40 + 4 call | | Reference method call | | Tag-It CFTR 40 + 4 call | | call | 40 + 4 call |
| 55 | 23786 | G542X | HET | G542X | HET | | | | | 7T/9T | 7T/9T D |
| 56 | 23811 | 2789 + 5G > A | HET | 2789 + 5G > A | HET | | | | | 7T | 7T D |
| 57^{a} | 24582 | — | ND | — | WT | | | | | 7T | 7T D |
| 58 | 24591 | 2789 + 5G > A | HET | 2789 + 5G > A | HET | ΔF508 | HET | ΔF508 | HET | 7T/9T | 7T/9T D |
| 59 | 24598 | R117H | HET | R117H | HET | ΔF508 | HET | ΔF508 | HET | 7T/9T | 7T/9T D |
| 60 | 25214 | 621 + 1G > T | HET | 621 + 1G > T | HET | | | | | 7T/9T | 7T/9T D |
| 61 | 25216 | 621 + 1G > T | HET | 621 + 1G > T | HET | | | | | 7T/9T | 7T/9T D |
| 62 | 25307 | 1717 − 1G > A | HET | 1717 − 1G > A | HET | ΔF508 | HET | ΔF508 | HET | 7T/9T | 7T/9T D |
| 63^{b} | 25362 | R347P | HET | R347P | HET | ΔF508 | ND/HET | ΔF508 | HET | 7T/9T | 7T/9T D |
| 64 | 26542 | 1717 − 1G > A | HET | 1717 − 1G > A | HET | | | | | 7T | 7T D |
| 65 | 27583 | R117H | HET | R117H | HET | ΔF508 | HET | ΔF508 | HET | 7T/9T | 7T/9T D |
| 66 | 27885 | R117H | HET | R117H | HET | ΔF508 | HET | ΔF508 | HET | 5T/9T | 5T/9T D |
| 67 | 29533 | R117H | HET | R117H | HET | 1898 + 1G > A | HET | 1898 + 1G > A | HET | 5T/7T | 5T/7T D |
| 68 | 29895 | R347P | HET | R347P | HET | ΔF508 | HET | ΔF508 | HET | 7T/9T | 7T/9T D |
| 69 | 30178 | ΔF508 | HET | ΔF508 | HET | 621 + 1G > T | HET | 621 + 1G > T | HET | 9T | 9T D |
| 70 | 30267 | ΔF508 | MUT | ΔF508 | Mu D | | | | | 9T | 9T D |
| 71 | 30445 | R117H | HET | R117H | HET | ΔF508 | HET | ΔF508 | HET | 5T/9T | 5T/9T D |
| 72 | 46013 | R553X | HET | R553X | HET | ΔF508 | HET | ΔF508 | HET | 7T/9T | 7T/9T D |

^{a}DNA sample 57 was called WT for all mutations and variants by both the ABI-CF System and by the CFTR 40 + 4 genotyping assay.
^{b}For the ΔF508 variant, DNA sample 63 was genotyped as WT by the ABI-CF System and HET by both DNA sequencing and the CFTR 40 + 4 genotyping assay.

TABLE 10
Genotyping calls obtained by the reference methods and the CFTR 40 + 4 genotyping
assay for the mutations/variants and T-tract of DNA Samples 73-92

| | | | | | | | | | | T-Tract variant | |
|---|---|---|---|---|---|---|---|---|---|---|---|
| | | | | | | | | | | Reference | Tag-It |
| Tm | HSC | Mutation/Variant 1 | | | | Mutation/Variant 2 | | | | method | CFTR |
| Sample # | Sample # | Reference method call | | Tag-It CFTR 40 + 4 call | | Reference method call | | Tag-It CFTR 40 + 4 call | | call | 40 + 4 call |
| 73 | 30460 | ΔI507 | HET | ΔI507 | HET | | | | | 7T | 7T D |
| 74 | 31157 | 1898 + 1G > A | HET | 1898 + 1G > A | HET | | | | | 7T | 7T D |
| 75 | 31391 | ΔF508 | HET | ΔF508 | HET | | | | | 7T/9T | 7T/9T D |
| 76 | 31815 | W1282X | MUT | W1282X | Mu D | | | | | 7T | 7T D |
| 77 | 31851 | ΔF508 | MUT | ΔF508 | Mu D | | | | | 9T | 9T D |
| 78 | 32032 | ΔF508 | MUT | ΔF508 | Mu D | | | | | 9T | 9T D |
| 79 | 32837 | ΔF508 | MUT | ΔF508 | Mu D | | | | | 9T | 9T D |
| 80 | 3284 | 1717 − 1G > A | HET | 1717 − 1G > A | HET | | | | | 7T | 7T D |
| 81 | 37435 | ΔF508 | MUT | ΔF508 | Mu D | | | | | 9T | 9T D |
| 82 | 46014 | 621 + 1G > T | HET | 621 + 1G > T | HET | 3120 + 1G > A | HET | 3120 + 1G > A | HET | 7T/9T | 7T/9T D |
| 83 | 37511 | ΔF508 | HET | ΔF508 | HET | W1282X | HET | W1282X | HET | 7T/9T | 7T/9T D |
| 84 | 37573 | ΔF508 | MUT | ΔF508 | Mu D | | | | | 9T | 9T D |
| 85 | 38165 | A455E | HET | A455E | HET | 621 + 1G > T | HET | 621 + 1G > T | HET | 9T | 9T D |
| 86 | 46015 | N1303K | HET | N1303K | HET | | | | | 7T/9T | 7T/9T D |
| 87 | 38435 | 1717 − 1G > A | HET | 1717 − 1G > A | HET | | | | | 7T | 7T D |
| 88 | 38801 | ΔF508 | MUT | ΔF508 | Mu D | | | | | 9T | 9T D |
| 89 | 3893 | 711 + 1G > T | HET | 711 + 1G > T | HET | ΔF508 | HET | ΔF508 | HET | 7T/9T | 7T/9T D |
| 90 | 3894 | 711 + 1G > T | HET | 711 + 1G > T | HET | | | | | 7T | 7T D |
| 91 | 3952 | 1717 − 1G > A | HET | 1717 − 1G > A | HET | | | | | 7T | 7T D |
| 92 | 40204 | ΔF508 | HET | ΔF508 | HET | G551D | HET | G551D | HET | 7T/9T | 7T/9T D |

TABLE 11
Genotyping calls obtained by the reference methods and the CFTR 40 + 4 genotyping
assay for the mutations/variants and T-tract of DNA Samples 93-112

| | | | | | | | | | | T-Tract variant | |
|---|---|---|---|---|---|---|---|---|---|---|---|
| Tm | HSC | | | | | | | | | Reference | Tag-It |
| Sample | Sample | Mutation/Variant 1 | | | | Mutation/Variant 2 | | | | method | CFTR |
| # | # | Reference method call | | Tag-It CFTR 40 + 4 call | | Reference method call | | Tag-It CFTR 40 + 4 call | | call | 40 + 4 call |
| 93 | 4073 | 3659delC | HET | 3659delC | HET | ΔF508 | HET | ΔF508 | HET | 7T/9T | 7T/9T D |
| 94 | 40801 | ΔF508 | HET | ΔF508 | HET | 3849 + 10kbC > T | HET | 3849 + 10kbC > T | HET | 7T/9T | 7T/9T D |
| 95 | 40805 | ΔF508 | HET | ΔF508 | HET | 3849 + 10kbC > T | HET | 3849 + 10kbC > T | HET | 7T/9T | 7T/9T D |
| 96 | 4085 | 3659delC | HET | 3659delC | HET | | | | | 7T | 7T D |
| 97^{a} | 40912 | ΔF508 | MUT | ΔF508 | Mu D | | | | | 7T/11T | 7T D |
| 98 | 41165 | ΔF508 | HET | ΔF508 | HET | 3849 + 10kbC > T | HET | 3849 + 10kbC > T | HET | 7T/9T | 7T/9T D |
| 99 | 41872 | G551D | HET | G551D | HET | ΔF508 | HET | ΔF508 | HET | 7T/9T | 7T/9T D |
| 100 | 42192 | R553X | HET | R553X | HET | ΔF508 | HET | ΔF508 | HET | 7T/9T | 7T/9T D |
| 101^{a} | 42232 | ΔF508 | MUT | ΔF508 | Mu D | | | | | 7T/11T | 7T D |
| 102 | 42234 | ΔF508 | MUT | ΔF508 | Mu D | | | | | 9T | 9T D |
| 103 | 42357 | ΔF508 | HET | ΔF508 | HET | R347P | HET | R347P | HET | 7T/9T | 7T/9T D |
| 104 | 4247 | N1303K | HET | N1303K | HET | ΔF508 | HET | ΔF508 | HET | 9T | 9T D |
| 105 | 4250 | N1303K | HET | N1303K | HET | | | | | 7T/9T | 7T/9T D |
| 106 | 42681 | ΔF508 | MUT | ΔF508 | Mu D | | | | | 9T | 9T D |
| 107 | 42705 | ΔF508 | MUT | ΔF508 | Mu D | | | | | 9T | 9T D |
| 108 | 42706 | ΔF508 | MUT | ΔF508 | Mu D | | | | | 9T | 9T D |
| 109 | 42903 | ΔF508 | HET | ΔF508 | HET | R117H | HET | R117H | HET | 7T/9T | 7T/9T D |
| 110 | 43189 | ΔF508 | HET | ΔF508 | HET | 3849 + 10kbC > T | HET | 3849 + 10kbC > T | HET | 7T/9T | 7T/9T D |
| 111 | 43337 | ΔF508 | MUT | ΔF508 | Mu D | | | | | 9T | 9T D |
| 112 | 43535 | ΔF508 | MUT | ΔF508 | Mu D | | | | | 9T | 9T D |

^{a}DNA samples 97 and was called 7T/11T for the 5T/7T/9T variant by DNA sequencing while the CFTR 40 + 4 genotyping assay only detected the 7T allele. The genotyping calls made by sequencing and the correcponding calls made by the CFTR 40 + 4 genotyping assay were removed from further data analysis.

TABLE 12
Genotyping calls obtained by the reference methods and the CFTR 40 + 4 genotyping
assay for the mutations/variants and T-tract of DNA Samples 113-131

| | | | | | | | | | | T-Tract variant | |
|---|---|---|---|---|---|---|---|---|---|---|---|
| Tm | | | | | | | | | | Reference | Tag-It |
| Sample | HSC | Mutation/Variant 1 | | | | Mutation/Variant 2 | | | | method | CFTR |
| # | Sample # | Reference method call | | Tag-It CFTR 40 + 4 call | | Reference method call | | Tag-It CFTR 40 + 4 call | | call | 40 + 4 call |
| 113 | 43607 | ΔF508 | HET | ΔF508 | HET | G542X | HET | G542X | HET | 9T | 9T D |
| 114 | 43759 | ΔF508 | HET | ΔF508 | HET | G551D | HET | G551D | HET | 7T/9T | 7T/9T D |
| 115 | 43957 | ΔF508 | HET | ΔF508 | HET | R117H | HET | R117H | HET | 5T/9T | 5T/9T D |
| 116 | 44846 | ΔF508 | HET | ΔF508 | HET | W1282X | HET | W1282X | HET | 7T/9T | 7T/9T D |
| 117 | 45633 | ΔF508 | HET | ΔF508 | HET | 1717 − 1G > A | HET | 1717 − 1G > A | HET | 7T/9T | 7T/9T D |
| 118 | 4917 | 1717 − 1G > A | HET | 1717 − 1G > A | HET | ΔF508 | HET | ΔF508 | HET | 7T/9T | 7T/9T D |
| 119 | 4919 | 1717 − 1G > A | HET | 1717 − 1G > A | HET | | | | | 7T | 7T D |
| 120 | 4995 | R553X | HET | R553X | HET | | | | | 7T | 7T D |
| 121 | 5001 | R553X | HET | R553X | HET | ΔF508 | HET | ΔF508 | HET | 7T/9T | 7T/9T D |
| 122 | 5109 | G542X | HET | G542X | HET | | | | | 7T/9T | 7T/9T D |
| 123 | 6322 | G551D | HET | G551D | HET | R117H | HET | R117H | HET | 7T | 7T D |
| 124 | 6340 | R117H | MUT | R117H | Mu D | | | | | 5T | 5T D |
| 125 | 7303 | N1303K | HET | N1303K | HET | | | | | 7T/9T | 7T/9T D |
| 126 | 7636 | N1303K | HET | N1303K | HET | | | | | 7T/9T | 7T/9T D |
| 127 | 7640 | G551D | HET | G551D | HET | | | | | 5T/7T | 5T/7T D |
| 128 | 7778 | R117H | HET | R117H | HET | | | | | 7T | 7T D |
| 129 | 8264 | G551D | HET | G551D | HET | ΔF508 | HET | ΔF508 | HET | 7T/9T | 7T/9T D |
| 130 | 8270 | G551D | HET | G551D | HET | | | | | 7T | 7T D |
| 131^{a} | 8411 | 621 + 1G > T | HET/ | 621 + 1G > T | WT | ΔF508 | ND/HET | ΔF508 | HET | 7T/9T | 7T/9T D |
| | | | WT | | | | | | | | |

^{a}For the 621 + 1G > A mutation, DNA sample 131 was genotyped as HET by the ABI-CF System and WT by both DNA sequencing and the CFTR 40 + 4 genotyping assay. For the ΔF508 variant, the same DNA sample was genotyped as WT by the ABI-CF System and HET by both DNA sequencing and the CFTR 40 + 4 genotyping assay.

TABLE 13
Genotyping calls obtained by the reference methods and the CFTR 40 + 4 genotyping
assay for the mutations/variants and T-tract of DNA Samples 132-141

| | | | | | | | | | | T-TRACT variant | |
|---|---|---|---|---|---|---|---|---|---|---|---|
| | | | | | | | | | | Reference | Tag-It |
| Tm | HSC | Mutation/Variant 1 | | | | Mutation/Variant 2 | | | | method | CFTR |
| Sample # | Sample # | Reference method call | | Tag-It CFTR 40 + 4 call | | Reference method call | | Tag-It CFTR 40 + 4 call | | call | 40 + 4 call |
| 132 | 8486 | G542X | HET | G542X | HET | ΔF508 | HET | ΔF508 | HET | 9T | 9T D |
| 133 | 8611 | G551D | HET | G551D | HET | ΔF508 | HET | ΔF508 | HET | 7T/9T | 7T/9T D |
| 134 | 9217 | 621 + 1G > T | HET | 621 + 1G > T | HET | ΔF508 | HET | ΔF508 | HET | 9T | 9T D |
| 135 | 9343 | G551D | HET | G551D | HET | | | | | 7T | 7T D |
| 136 | 9354 | G542X | HET | G542X | HET | R117H | HET | R117H | HET | 5T/9T | 5T/9T D |
| 137^{a} | 9376 | G551D | HET | G551D | HET | R334W | ND/HET | R334W | HET | 7T | 7T D |
| 138 | 9500 | G551D | HET | G551D | HET | ΔF508 | HET | ΔF508 | HET | 7T/9T | 7T/9T D |
| 139 | 9542 | N1303K | HET | N1303K | HET | | | | | 5T/7T | 5T/7T D |
| 140 | 9770 | 2789 + 5G > A | HET | 2789 + 5G > A | HET | ΔF508 | HET | ΔF508 | HET | 7T/9T | 7T/9T D |
| 141 | 9854 | 1717 − 1G > A | HET | 1717 − 1G > A | HET | ΔF508 | HET | ΔF508 | HET | 7T/9T | 7T/9T D |

^{a}For the R334W mutation, DNA sample 137 was genotyped as WT by the ABI-CF System and HET by both DNA sequencing and the CFTR 40 + 4 genotyping assay.

For Tables 6 to 13, unless indicated, all other mutations and variants were called WT (i.e.: the mutant allele was not detected). For all samples (except for sample 15), no more than two mutation or variants in each were non-WT.

The genotyping calls obtained from the reference methods (DNA sequencing and ABI-CF System) are found in Tables 6-13. Of the 44 mutations and variants detected and simultaneously genotyped by the CFTR 40+4 genotyping assay, the ABI-CF System can genotype 30 of these mutations and variants. The remaining 14 mutations and variants were genotyped by Genaissance Pharmaceuticals via DNA sequencing. The genotyping calls obtained from the ABI-CF System are summarized in Table 14.

TABLE 14

| Mutation/Variant | WT | HET | MUT |
|---|---|---|---|
| G85E | 138 | 1 | 0 |
| R117H | 126 | 12 | 1 |
| Y122X | 139 | 0 | 0 |
| 621 + 1G > T | 131 | 8 | 0 |
| 711 + 1G > T | 136 | 3 | 0 |
| 1078delT | 138 | 1 | 0 |
| R334W | 133 | 4 | 2 |
| R347P | 136 | 3 | 0 |
| R347H | 139 | 0 | 0 |
| A455E | 138 | 1 | 0 |
| ΔI507 | 137 | 2 | 0 |
| ΔF508 | 69 | 55 | 15 |
| V520F | 139 | 0 | 0 |
| 1717-1G > A | 130 | 9 | 0 |
| G542X | 128 | 11 | 0 |
| S549N | 139 | 0 | 0 |
| S549R | 139 | 0 | 0 |
| G55ID | 128 | 11 | 0 |
| R553X | 135 | 4 | 0 |
| R560T | 133 | 6 | 0 |
| 1898 + 1G > A | 137 | 2 | 0 |
| 2183AA > G | 139 | 0 | 0 |
| 2789 + 5G > A | 134 | 5 | 0 |
| R1162X | 135 | 4 | 0 |
| 3659delC | 134 | 5 | 0 |
| 3849 + 10kbC > T | 130 | 9 | 0 |
| 3905insT | 138 | 1 | 0 |
| W1282X | 127 | 11 | 1 |
| N1303K | 130 | 9 | 0 |
| F508C | 139 | 0 | 0 |

^{a}The genotyping calls were provided by Dr. Peter Ray's laboratory and were based on those mutations and variants detected by the ABI-CF System. If the ABI-CF System did not detect either the heterozygous (HET) or mutant (MUT) genotype, the mutation or variant was considered to be wild-type (WT) for that DNA sample.

Results from the Reference Method of DNA Sequencing

The initial DNA sequencing reactions performed by Genaissance Pharmaceuticals produced a requirement for several sequencing repeats (Table 15) due to the absence of successful sequencing either in one or in both DNA sequencing directions.

TABLE 15

| | | | Number of genotyping call failures | |
|---|---|---|---|---|
| | Exon | Mutation/Variant | Failed calls^{a} | Total failed |
| Initial sequencing | Exon 3 | 394delTT | 13 | 482 |
| | Exon 4 | I148T | 14 | |
| | Exon 9 | 5T/7T/9T | 33 | |

| | | | |
|---|---|---|---|
| Exon 10 | I506V | 69 | (70 unique |
| | I507V | 22 | DNA samples) |

| | | |
|---|---|---|
| Exon 11 | A559T | 5 |
| Exon 12 | 1898 + 5G > T | 3 |
| Exon 13^{b} | 2184delA | 0 |
| | 2307insA | |
| Exon 16 | 3120 + 1G > A | 25 |

| | | | |
|---|---|---|---|
| Exon 17b | Y1092X | 91 | (91 unique |
| | M1101K | 75 | DNA samples) |

| | | |
|---|---|---|
| Exon 19 | S1255X | 76 |

| | | | |
|---|---|---|---|
| Exon 20 | S1255X | 46 | (47 unique |
| | 3876delA | 35 | DNA samples) |

| | | | | |
|---|---|---|---|---|
| Sequencing repeat^{c} | Exon 3 | 394delTT | 0 | 37 |
| | Exon 4 | I148T | 0 | |
| | Exon 9 | 5T/7T/9T | 0 | |
| | Exon 10 | I506V | 0 | |
| | | I507V | | |
| | Exon 11 | A559T | 0 | |
| | Exon 12 | 1898 + 5G > T | 0 | |
| | Exon 13^{b} | 2184delA | 0 | |
| | | 2307insA | | |
| | Exon 16 | 3120 + 1G > A | 3 | |

| | | | |
|---|---|---|---|
| Exon 17b | Y1092X | 12 | (20 unique |
| | M1101K | 20 | DNA samples) |

| | | |
|---|---|---|
| Exon 19 | S1255X | 2 |
| Exon 20 | S1255X | 0 |
| | 3876delA | |

^{a}For the exons containing two mutations (i.e.: exons 10, 13 and 20), it was possible that either one of the pair or both of the mutations could not be genotyped from the same exon. In these cases the total number of actual genotyping call failures was indicated as the total number of call failures (e.g. if mutation Y1092X and M1101K of sample 1 could not be genotyped, both mutations in the same exon were counted as individual call failures).

Table 15 presents the number of genotyping call failures observed from the initial DNA sequencing run and in the DNA sequencing repeats (the 14 mutations and variants are indicated) for each of the 139 DNA samples tested.

DNA samples which failed to give unambiguous genotyping calls for a particular mutation or variant in both directions were resequenced in both directions. Repeat sequencing was performed on the same amplimers. The only amplimers that required reamplification were for exon 13 (see below).

Since there were many initial genotyping failures observed for some of the exons, it was more convenient for Genaissance Pharmaceuticals to repeat the sequencing for all samples for that exon.

Exons 11 (mutation A559T) and 12 (mutation 1898+5G>T) required a small number repeat DNA sequencing (5 and 3 respectively) and thus only those DNA samples which failed to be genotyped for their mutation were resequenced (samples 77, 78, 89, 91 and 114 for exon 11 and samples 13, 62 and 134 for exon 12). In exons 3 (mutation 394delTT) and 4 (mutation 1148T), the DNA samples which required a sequencing repeat were spread throughout the 139 DNA samples so much so that it was more convenient to repeat the sequencing of the samples, rather than repeat the sequencing for those specific samples. When analyzing the DNA sequencing data from the repeats, it was only the samples which failed that were investigated. The DNA sequencing electropherograms of exon 13 indicated contamination of the opposite strand in the sequencing data of both the forward and reverse DNA sequencing reactions. Because of this, it was decided to prepare fresh amplimers of exon 13 for each DNA sample. These new amplimers were shipped to Genaissance Pharmaceuticals for sequencing. The DNA sequencing did not indicate any contamination; moreover, most of genotyping calls could be made from this reaction. A small number did require resequencing but genotyping calls were made based on the DNA sequencing results from a single direction. DNA sample 14 was genotyped as WT for the 2307insA mutation from the reverse sequencing direction only and DNA sample 74 was genotyped as WT for the 2184delA mutation from the forward sequencing direction only.

For the case of the T-tract variants (detected by the CFTR 40+4 genotyping assay as the 5T, 7T and 9T alleles), the final genotyping calls obtained from DNA sequencing are summarized in Tables 6 to 13. The DNA sequencing called samples 97 and 101 each as a 7T/11T genotype. Since the CFTR 40+4 genotyping assay cannot detect the 11T allele, these two calls were eliminated from further comparison.

Of the genomic DNA samples that required the DNA sequencing to be repeated, 37 genotyping calls could not be genotyped in the end. The 37 failed genotyping calls were therefore removed from further analysis. Two calls from the T-tract were also removed from the set of genotyping calls obtained from the reference methods that were compared against the CFTR 40+4 genotyping assay. A total of 6077 genotyping calls (i.e.: 6116 total expected genotypic calls −37 mutations/variants that could not be called −2 calls that could not be detected by the CFTR 40+4 genotyping assay) were thus compared against the corresponding genotyping calls obtained by the CFTR 40+4 genotyping assay.

Table 16 summarizes the total number of samples that, through DNA sequencing, were called as either WT, HET, or MUT for each mutation/variant tested for. This table accounts for the removal of all the individual genotyping call failures from DNA sequencing (see above).

In general, the DNA sequencing results showed that only DNA sample 82 was called as heterozygous (HET) for mutation 3120+1G>A (found in exon 16), all other mutations and variants that were successfully sequenced were called WT.

CFTR 40+4 Genotyping Assay Results

The calls made by the software used in the method of the present invention are found in Tables 6 to 13. The genomic DNA samples were initially divided into seven batches, each contained up to 23 samples and one negative control. All 139 DNA samples analyzed with the CFTR 40+4 genotyping assay successfully provided genotypes without the need for reruns.

DNA Sample Reanalysis Due to Discordance

Up until this point, any assay repeats performed were for obtaining unambiguous genotyping calls for all mutations or variants of each DNA sample. After all required DNA sequencing repeats, and the removal of mutations or variants that could not be genotyped, the remaining unambiguous calls were analyzed. The genotyping calls determined by the CFTR 40+4 genotyping assay were then compared to the corresponding genotyping calls obtained by the reference methods. When these calls were initially compared, eight discordant calls in seven DNA samples were identified (see below). The DNA samples were reanalyzed by DNA sequencing in order to resolve the discordances. Upon the reanalysis, seven of these eight discordant calls from the CFTR 40+4 genotyping assay were resolved and found to be concordant to the corresponding calls obtained by DNA sequencing.

TABLE 16

| Mutation/Variant | WT | HET | MUT | Notes |
|---|---|---|---|---|
| 394delTT | 139 | 0 | 0 | — |
| I148T | 139 | 0 | 0 | — |
| I506V | 139 | 0 | 0 | 1 sample called WT from |
| | | | | forward sequence only |
| I507V | 139 | 0 | 0 | 3 samples called WT from |
| | | | | forward sequence only |
| A559T | 139 | 0 | 0 | — |
| 1898 + 5G > T | 139 | 0 | 0 | — |
| 2184delA | 139 | 0 | 0 | 1 sample called WT from |
| | | | | forward sequence only |
| 2307insA | 139 | 0 | 0 | 1 sample called WT from |
| | | | | reverse sequence only |
| 3120 + 1G > A | 135 | 1 | 0 | 5 samples called WT from |
| | | | | forward sequence only. |
| | | | | 3 samples failed to be |
| | | | | genotyped |
| Y1092X | 127 | 0 | 0 | 1 sample called WT from |
| (C > G or C > A) | | | | forward sequence only |
| | | | | 3 samples called WT from |
| | | | | reverse sequence only |
| | | | | 12 samples failed to be |
| | | | | genotyped |
| M1101K | 119 | 0 | 0 | 2 samples called WT from |
| | | | | forward sequence only |
| | | | | 20 samples called WT from |
| | | | | reverse sequence only |
| | | | | 20 samples failed to be |
| | | | | genotyped |
| S1255X (exon 19) | 137 | 0 | 0 | 2 samples called WT from |
| | | | | forward sequence only |
| | | | | 25 samples called WT from |
| | | | | reverse sequence only |
| | | | | 2 samples failed to be |
| | | | | genotyped |
| S1255X (exon 20) | 139 | 0 | 0 | 2 samples called WT from |
| | | | | forward sequence only |
| | | | | 3 samples called WT from |
| | | | | reverse sequence only |
| 3876delA | 139 | 0 | 0 | 1 sample called WT from |
| | | | | forward sequence only |
| | | | | 1 sample called WT from |
| | | | | reverse sequence only |

DNA sample 15 was genotyped by the ABI-CF System as heterozygous (HET) for the R560T mutation (found in exon 11). The CFTR 40+4 genotyping assay called this sample as wild-type (WT). Results from the DNA sequencing of exon 11 indicated a wild-type (WT) genotype for this sample, which was concordant to the CFTR 40+4 genotyping assay.
DNA sample 36 was genotyped by the ABI-CF System as WT (i.e.: a mutant allele was not detected) for the 1898+1G>A mutation (found in exon 12). The CFTR 40+4 genotyping assay called this sample as HET. Results from the DNA sequencing of exon 12 indicated a HET genotype for this sample, which was concordant to the CFTR 40+4 genotyping assay.
DNA sample 37 was genotyped by the ABI-CF System as WT (i.e.: a mutant allele was not detected) for the 1898+1G>A mutation. The CFTR 40+4 genotyping assay called this sample as HET. The DNA sequencing results from exon 12 indicated a HET genotype for sample 37, which was concordant to the CFTR 40+4 genotyping assay.
DNA sample 38 was genotyped by the ABI-CF System as HET for the ΔF508 mutation (found in exon 10). The CFTR 40+4 genotyping assay called this sample as WT, a result which was concordant to the DNA sequencing results of exon 10.
DNA sample 72 was genotyped by the ABI-CF System as WT (i.e.: a mutant allele was not detected) for the ΔF508 mutation. The CFTR 40+4 genotyping assay called this sample as HET. Analysis of the DNA sequencing results for exon 10 indicated a HET genotype for this sample, a result which was concordant to the CFTR 40+4 genotyping assay.
DNA sample 131 was genotyped by the ABI-CF System as WT (i.e.: a mutant allele was not detected) for the 621+1G>T mutation (found in exon 4). The CFTR 40+4 genotyping assay called this sample as HET. Analysis of the DNA sequencing of exon 4 indicated a HET genotype for this sample, a result which was concordant to the CFTR 40+4 genotyping assay. This sample was also genotyped by the ABI-CF System as WT for the ΔF508 mutation. The CFTR 40+4 genotyping assay called this sample as HET and DNA sequencing analysis of exon 10 of this sample indicated a HET genotype, a result which was concordant to the CFTR 40+4 genotyping assay.
DNA sample 137 was genotyped by the ABI-CF System as WT (i.e.: a mutant allele was not detected) for the R334W mutation. The CFTR 40+4 genotyping assay called this sample as HET. This mutation was found in exon 7. In order to resolve this discordance sample 137 was amplified for exon 7 by the PCR and then sequenced. Subsequent DNA sequencing analysis of exon 7 of this sample indicated a HET genotype, a result which was concordant to the CFTR 40+4 genotyping assay.

Analysis of the T-Tract Variant (5T/7T/9T) in Exon 9

Samples 97 and 101 were called by DNA sequencing as a 7T/11T genotype for the T-tract variant found in exon 9. The CFTR 40+4 genotyping assay detected the 7T allele for these two samples. Though not necessarily discordant calls, what was evident was the ability of the CFTR 40+4 genotyping assay to successfully detect the 7T allele and inability of the CFTR 40+4 genotyping assay to detect the 11T allele. These two calls were thus mutually eliminated from comparison between the reference methods of genotyping and the CFTR 40+4 genotyping assay. All other DNA samples exhibited concordant genotyping calls between the CFTR 40+4 genotyping assay and DNA sequencing reference method for the T-tract variant.

Mutation/Variant Call Percent Concordance and Kit Overall Percent Concordance

The call accuracy of the CFTR 40+4 genotyping assay, as measured by percent concordance to the reference methods of genotyping was determined for each of the 44 mutations and variants detected by the CFTR 40+4 genotyping assay. The final results indicated >97% concordance of the CFTR 40+4 genotyping assay to the reference methods except for those discordances indicated above. In these cases though, the percent concordances for the affected mutation or variant remained greater than 95%. The percent concordances for these affected mutations and variants are indicated in Table 9.

After the allowed reanalysis of the ABI-CF System results via DNA sequencing by Genaissance Pharmaceuticals, the percent concordance increased for these mutations and variants (see Table 17).

Table 17 presents the percent concordance between the genotyping calls obtained by the CFTR 40+4 genotyping assay and the reference methods prior to and after reanalysis of available DNA sequencing data.

TABLE 17

| Mutation/ | Number of | Initial % | % concordance |
|---|---|---|---|
| Variant | discordant calls | concordance | after reanalysis |
| ΔF508 | 3 | 97.8 | 100 |
| | (Samples 38, 72 and 131) | | |
| 1898 + 1G > A | 2 | 98.5 | 100 |
| | (Samples 36 and 37) | | |
| R560T | 1 | 99.3 | 100 |
| | (Sample 15) | | |
| 621 + 1G > T | 1 | 99.3 | 100 |
| | (Sample 131) | | |
| R334W | 1 | 99.3 | 100 |
| | (Sample 137) | | |

In summary, prior to and after the reanalysis of discordant DNA samples, the overall CFTR 40+4 genotyping assay percent concordance to the reference methods was greater than the minimal 98% acceptance criteria. The initial overall CFTR 40+4 genotyping assay percent concordance was 99.87% (eight discordances). After reanalysis, the CFTR 40+4 genotyping assay percent concordance was 100%.

Call Rate Comparison

It was also useful to be able to compare the call rate of the CFTR 40+4 genotyping assay to the call rate of the reference methods. This comparison did not address whether there was concordance in the calls but specifically the ability of the genotyping methods to successfully yield calls. Since the rerun rate for the non-sequencing genotyping methods was not known, the call rate of the reference methods was derived from the call rate of the DNA sequencing. To determine the call rate of the CFTR 40+4 genotyping assay and the reference method of DNA sequencing, the number of expected calls was determined from the final number of DNA samples tested equally by both methods for the 14 mutations and variants indicated in Table 5 Therefore, there were 1946 possible genotyping calls to be made on the 139 DNA samples that were tested by both methods.

The reference method of DNA sequencing had a call rate of 75% from a single initial sequencing run (482 failed calls that required sequencing repeats). After the sequencing repeats, 37 genotypes still failed to be called. Thus, sequencing of 14 mutations and variants for each of 139 DNA samples produced a final call rate of 98%, (see Table 18) for DNA sequencing. The final call rate for DNA sequencing even allowed for some samples to be called from DNA sequencing data obtained confidently from only one sequencing direction.

In the case of the CFTR 40+4 genotyping assay, the initial assay runs resulted in the successful calling of all 139 DNA samples yielding a call rate of 100% and no requirement for a repeat of the assay, (see Table 18). It should be noted that all 37 genotyping calls that could not be made unambiguously by DNA sequencing were called by the CFTR 40+4 genotyping assay as WT.

TABLE 18
Call Rates of the CFTR 40 + 4 Genotyping Assay and DNA
Sequencing Based on 14 Mutations and Variants That Were
Tested by Both Methods

Call Rate

| Genotyping method | Initial | After repeats |
|---|---|---|
| DNA Sequencing | 75% | 98% |
| CFTR 40 + 4 Genotyping Assay | 100% | NA |

Although the invention has been described with reference to certain specific embodiments, various modifications thereof will be apparent to those skilled in the art without departing from the spirit and scope of the invention as outlined in the claims appended hereto.

### REFERENCES

(1990). “Gradient of distribution in Europe of the major CF mutation and of its associated haplotype. European Working Group on CF Genetics (EWGCFG).” Hum Genet. 85(4): 436-45.
(1997). “Genetic Testing for Cystic Fibrosis.” NIH Consensus Statement 15(4): 1-37.
(1999-2004). The Merck Manual of Diagnosis and Therapy.
Abeliovich, D., I. P. Lavon, et al. (1992). “Screening for five mutations detects 97% of cystic fibrosis (CF) chromosomes and predicts a carrier frequency of 1:29 in the Jewish Ashkenazi population.” Am J Hum Genet. 51(5): 951-6.
al-Jader, L. N., A. L. Meredith, et al. (1992). “Severity of chest disease in cystic fibrosis patients in relation to their genotypes.” J Med Genet. 29(12): 883-7.
Alper, O. M., S. G. Shu, et al. (2003). “Detection of novel CFTR mutations in Taiwanese cystic fibrosis patients.” J Formos Med Assoc 102(5): 287-91.
Alton, E. W., M. Stem, et al. (1999). “Cationic lipid-mediated CFTR gene transfer to the lungs and nose of patients with cystic fibrosis: a double-blind placebo-controlled trial.” Lancet 353(9157): 947-54.
Anderson, M. P., H. A. Berger, et al. (1991). “Nucleoside triphosphates are required to open the CFTR chloride channel.” Cell 67(4): 775-84.
Antinolo, G., S. Borrego, et al. (1997). “Genotype-phenotype relationship in 12 patients carrying cystic fibrosis mutation R334W.” J Med Genet. 34(2): 89-91.
Auerbach, H. S., M. Williams, et al. (1985). “Alternate-day prednisone reduces morbidity and improves pulmonary function in cystic fibrosis.” Lancet 2(8457): 686-8.
Augarten, A., B. S. Kerem, et al. (1993). “Mild cystic fibrosis and normal or borderline sweat test in patients with the 3849+10 kb. C-->T mutation.” Lancet 342(8862): 25-6.
Banjar, H., M. Kambouris, et al. (1999). “Geographic distribution of cystic fibrosis transmembrane regulator gene mutations in Saudi Arabia.” Ann Trop Paediatr 19(1): 69-73.
Bear, C. E., C. H. Li, et al. (1992). “Purification and functional reconstitution of the cystic fibrosis transmembrane conductance regulator (CFTR).” Cell 68(4): 809-18.
Bienvenu, T., S. Bousquet, et al. (1993). “Simultaneous detection of the two prevalent mutations in the cystic fibrosis gene in Reunion Island.” Hum Mutat 2(4): 306-8.
Bienvenu, T., F. Cartault, et al. (1996). “A splicing mutation in intron 16 of the cystic fibrosis transmembrane conductance regulator gene, associated with severe disease, is common on Reunion Island.” Hum Hered 46(3): 168-71.
Bozon, D., J. Zielenski, et al. (1994). “Identification of four new mutations in the cystic fibrosis transmembrane conductance regulator gene: 1148T, L1077P, Y1092X, 2183AA-->G.” Hum Mutat 3(3): 330-2.
Casals, T., V. Nunes, et al. (1993). “Cystic fibrosis in Spain: high frequency of mutation G542X in the Mediterranean coastal area.” Hum Genet. 91(1): 66-70.
Cashman, S. M., A. Patino, et al. (1995). “The Irish cystic fibrosis database.” J Med Genet. 32(12): 972-5.
Castaldo, G., A. Fuccio, et al. (1999). “Detection of five rare cystic fibrosis mutations peculiar to Southern Italy: implications in screening for the disease and phenotype characterization for patients with homozygote mutations.” Clin Chem 45(7): 957-62.
Chang, X. B., Y. X. Hou, et al. (1994). “Mapping of cystic fibrosis transmembrane conductance regulator membrane topology by glycosylation site insertion.” J Biol Chem 269(28): 18572-5.
Cheadle, J., L. al-Jader, et al. (1992). “Mild pulmonary disease in a cystic fibrosis child homozygous for R553X.” J Med Genet. 29(8): 597.
Cheadle, J., J. Myring, et al. (1992). “Mutation analysis of 184 cystic fibrosis families in Wales.” J Med Genet. 29(9): 642-6.
Cheng, S. H., R. J. Gregory, et al. (1990). “Defective intracellular transport and processing of CFTR is the molecular basis of most cystic fibrosis.” Cell 63(4): 827-34.
Cheng, S. H., D. P. Rich, et al. (1991). “Phosphorylation of the R domain by cAMP-dependent protein kinase regulates the CFTR chloride channel.” Cell 66(5): 1027-36.
Chevalier-Porst, F., A. M. Bonardot, et al. (1994). “Mutation analysis in 600 French cystic fibrosis patients.” J Med Genet. 31(7): 541-4.
Chiang, C. E., S. A. Chen, et al. (1997). “Genistein directly induces cardiac CFTR chloride current by a tyrosine kinase-independent and protein kinase A-independent pathway in guinea pig ventricular myocytes.” Biochem Biophys Res Commun 235(1): 74-8.
Chillon, M., T. Casals, et al. (1995). “Mutations in the cystic fibrosis gene in patients with congenital absence of the vas deferens.” N Engl J Med 332(22): 1475-80.
Chu, C. S., B. C. Trapnell, et al. (1993). “Genetic basis of variable exon 9 skipping in cystic fibrosis transmembrane conductance regulator mRNA.” Nat Genet. 3(2): 151-6.
Claustres, M., M. Desgeorges, et al. (1992). “Analysis of 30 known cystic fibrosis mutations: 10 mutations account for 27% of non-delta F508 chromosomes in southern France.” Hum Genet. 90(4): 464-6.
Cohen, B. E., G. Lee, et al. (1997). “8-cyclopentyl-1,3-dipropylxanthine and other xanthines differentially bind to the wild-type and delta F508 first nucleotide binding fold (NBF-1) domains of the cystic fibrosis transmembrane conductance regulator.” Biochemistry 36(21): 6455-61.
Corey, M., F. J. McLaughlin, et al. (1988). “A comparison of survival, growth, and pulmonary function in patients with cystic fibrosis in Boston and Toronto.” J Clin Epidemiol 41(6): 583-91.
Coutelle, C., R. Bruckner, et al. (1992). “Prevalence of cystic fibrosis mutations in the East German population.” Hum Mutat 1(2): 109-12.
Cutting, G. R., W. W. Grody, et al. (2004). 2004 Revision to ACMG's CFTR Carrier Screening Mutation Panel: A Preview.
Cutting, G. R., L. M. Kasch, et al. (1990). “Two patients with cystic fibrosis, nonsense mutations in each cystic fibrosis gene, and mild pulmonary disease.” N Engl J Med 323(24): 1685-9.
Cutting, G. R., L. M. Kasch, et al. (1990). “A cluster of cystic fibrosis mutations in the first nucleotide-binding fold of the cystic fibrosis conductance regulator protein.” Nature 346(6282): 366-9.
De Braekeleer, M., C. Allard, et al. (1997). “Genotype-phenotype correlation in cystic fibrosis patients compound heterozygous for the A455E mutation.” Hum Genet. 101(2): 208-11.
De Braekeleer, M., C. Allard, et al. (1998). “Phenotypic variability in five cystic fibrosis patients compound heterozygous for the Y1092X mutation.” Hum Hered 48(3): 158-62.
De Braekeleer, M., F. Simard, et al. (1997). “Little phenotypic variability in three CF sibs compound heterozygous for the 621+1G-->T and the 711+1G-->T mutations.” Clin Genet. 51(3): 214-6.

Dean, M., M. B. White, et al. (1990). “Multiple mutations in highly conserved residues are found in mildly affected cystic fibrosis patients.” Cell 61(5): 863-70.
Eidelman, O., C. Guay-Broder, et al. (1992). “A1 adenosine-receptor antagonists activate chloride efflux from cystic fibrosis cells.” Proc Natl Acad Sci USA 89(12): 5562-6.
Eigen, H., B. J. Rosenstein, et al. (1995). “A multicenter study of alternate-day prednisone therapy in patients with cystic fibrosis. Cystic Fibrosis Foundation Prednisone Trial Group.” J Pediatr 126(4): 515-23.
el-Harith, E. A., T. Dork, et al. (1997). “Novel and characteristic CFTR mutations in Saudi Arab children with severe cystic fibrosis.” J Med Genet. 34(12): 996-9.
Estivill, X., C. Bancells, et al. (1997). “Geographic distribution and regional origin of 272 cystic fibrosis mutations in European populations. The Biomed CF Mutation Analysis Consortium.” Hum Mutat 10(2): 135-54.
Estivill, X., L. Ortigosa, et al. (1995). “Clinical characteristics of 16 cystic fibrosis patients with the missense mutation R334W, a pancreatic insufficiency mutation with variable age of onset and interfamilial clinical differences.” Hum Genet. 95(3): 331-6.
Feldmann, D., R. Couderc, et al. (2003). “CFTR genotypes in patients with normal or borderline sweat chloride levels.” Hum Mutat 22(4): 340.
Ferec, C., C. Verlingue, et al. (1993). “Genotype analysis of adult cystic fibrosis patients.” Hum Mol Genet. 2(10): 1557-60.
Flodrops, H., M. Renouil, et al. (2003). “[Clinical aspects and genetic specificities of cystic fibrosis in Reunion Island].” Arch Pediatr 10(11): 955-9.
Fonknechten, N., J. C. Chomel, et al. (1992). “Skipping of exon 5 as a consequence of the 711+1 G-->T mutation in the CFTR gene.” Hum Mol Genet. 1(4): 281-2.
Frossard, P. M., E. Girodon, et al. (1998). “Identification of cystic fibrosis mutations in the United Arab Emirates. Mutations in brief no. 133. Online.” Hum Mutat 11(5): 412-3.
Frossard, P. M., J. Hertecant, et al. (1999). “Genotype-phenotype correlations in cystic fibrosis: clinical severity of mutation S549R(T-->G).” Eur Respir J 13(1): 100-2.
Fuchs, H. J., D. S. Borowitz, et al. (1994). “Effect of aerosolized recombinant human DNase on exacerbations of respiratory symptoms and on pulmonary function in patients with cystic fibrosis. The Pulmozyme Study Group.” N Engl J Med 331(10): 637-42.
Fulmer, S. B., E. M. Schwiebert, et al. (1995). “Two cystic fibrosis transmembrane conductance regulator mutations have different effects on both pulmonary phenotype and regulation of outwardly rectified chloride currents.” Proc Natl Acad Sci USA 92(15): 6832-6.
Gabriel, S. E., L. L. Clarke, et al. (1993). “CFTR and outward rectifying chloride channels are distinct proteins with a regulatory relationship.” Nature 363(6426): 263-8.
Gan, K. H., H. J. Veeze, et al. (1995). “A cystic fibrosis mutation associated with mild lung disease.” N Engl J Med 333(2): 95-9.
Gasparini, P., G. Borgo, et al. (1992). “Nine cystic fibrosis patients homozygous for the CFTR nonsense mutation R1162× have mild or moderate lung disease.” J Med Genet. 29(8): 558-62.
Gasparini, P., C. Marigo, et al. (1993). “Screening of 62 mutations in a cohort of cystic fibrosis patients from north eastern Italy: their incidence and clinical features of defined genotypes.” Hum Mutat 2(5): 389-94.
Gasparini, P., V. Nunes, et al. (1991). “The search for south European cystic fibrosis mutations: identification of two new mutations, four variants, and intronic sequences.” Genomics 10(1): 193-200.
Gibson, R., S. Moskowitz, et al. (2001). “Cystic Fibrosis.” Gene Reviews.
Gilbert, F., Z. Li, et al. (1995). “Clinical spectrum in homozygotes and compound heterozygotes inheriting cystic fibrosis mutation 3849+10 kbC>T: significance for geneticists.” Am Med Genet. 58(4): 356-9.
Goldman, A., R. Labrum, et al. (2001). “The molecular basis of cystic fibrosis in South Africa.” Clin Genet. 59(1): 37-41.
Grebe, T. A., W. W. Doane, et al. (1992). “Mutation analysis of the cystic fibrosis transmembrane regulator gene in Native American populations of the southwest.” Am Hum Genet. 51(4): 736-40.
Grebe, T. A., W. K. Seltzer, et al. (1994). “Genetic analysis of Hispanic individuals with cystic fibrosis.” Am J Hum Genet. 54(3): 443-6.
Grody, W. W., G. R. Cutting, et al. (2001). “Laboratory standards and guidelines for population-based cystic fibrosis carrier screening.” Genet Med 3(2): 149-54.
Hamilton, J. W. (2001). “Gentamicin in pharmacogenetic approach to treatment of cystic fibrosis.” Lancet 358(9298): 2014-6.
Hamosh, A., S. C. Fitz-Simmons, et al. (1998). “Comparison of the clinical manifestations of cystic fibrosis in black and white patients.” J Pediatr 132(2): 255-9.
Hamosh, A., B. C. Trapnell, et al. (1991). “Severe deficiency of cystic fibrosis transmembrane conductance regulator messenger RNA carrying nonsense mutations R553X and W1316X in respiratory epithelial cells of patients with cystic fibrosis.” J Clin Invest 88(6): 1880-5.
Heim, R. A., E. A. Sugarman, et al. (2001). “Improved detection of cystic fibrosis mutations in the heterogeneous U.S. population using an expanded, pan-ethnic mutation panel.” Genet Med 3(3): 168-76.
Hergersberg, M., J. Balakrishnan, et al. (1997). “A new mutation, 3905insT, accounts for 4.8% of 1173 CF chromosomes in Switzerland and causes a severe phenotype.” Hum Genet. 100(2): 220-3.
Highsmith, W. E., L. H. Burch, et al. (1994). “A novel mutation in the cystic fibrosis gene in patients with pulmonary disease but normal sweat chloride concentrations.” N Engl J Med 331(15): 974-80.
Highsmith, W. E., Jr., L. H. Burch, et al. (1997). “Identification of a splice site mutation (2789+5 G>A) associated with small amounts of normal CFTR mRNA and mild cystic fibrosis.” Hum Mutat 9(4): 332-8.
Hofmann, T., M. J. Stutts, et al. (1998). “Effects of topically delivered benzamil and amiloride on nasal potential difference in cystic fibrosis.” Am J Respir Crit. Care Med 157(6 Pt 1): 1844-9.
Howell, L. D., R. Borchardt, et al. (2000). “ATP hydrolysis by a CFTR domain: pharmacology and effects of G551D mutation.” Biochem Biophys Res Commun 271(2): 518-25.
Hull, J., S. Shackleton, et al. (1993). “Abnormal mRNA splicing resulting from three different mutations in the CFTR gene.” Hum Mol Genet. 2(6): 689-92.
Hull, J., S. Shackleton, et al. (1994). “The stop mutation R553X in the CFTR gene results in exon skipping.” Genomics 19(2): 362-4.
Jones, C. T., I. McIntosh, et al. (1992). “Three novel mutations in the cystic fibrosis gene detected by chemical cleavage: analysis of variant splicing and a nonsense mutation.” Hum Mol Genet 1(1): 11-7.
Kanavakis, E., A. Efthymiadou, et al. (2003). “Cystic fibrosis in Greece: molecular diagnosis, haplotypes, prenatal diagnosis and carrier identification amongst high-risk individuals.” Clin Genet. 63(5): 400-9.
Kere, J., X. Estivill, et al. (1994). “Cystic fibrosis in a low-incidence population: two major mutations in Finland.” Hum Genet. 93(2): 162-6.
Kerem, B., J. M. Rommens, et al. (1989). “Identification of the cystic fibrosis gene: genetic analysis.” Science 245(4922): 1073-80.
Kerem, B. S., J. Zielenski, et al. (1990). “Identification of mutations in regions corresponding to the two putative nucleotide (ATP)-binding folds of the cystic fibrosis gene.” Proc Natl Acad Sci USA 87(21): 8447-51.
Kerem, E., M. Corey, et al. (1990). “The relation between genotype and phenotype in cystic fibrosis—analysis of the most common mutation (delta F508).” N Engl J Med 323(22): 1517-22.
Kerem, E., Y. M. Kalman, et al. (1995). “Highly variable incidence of cystic fibrosis and different mutation distribution among different Jewish ethnic groups in Israel.” Hum Genet. 96(2): 193-7.
Kerem, E., M. Nissim-Rafinia, et al. (1997). “A missense cystic fibrosis transmembrane conductance regulator mutation with variable phenotype.” Pediatrics 100(3): E5.
Kessler, D., C. Moehlenkamp, et al. (1996). “Determination of cystic fibrosis carrier frequency for Zuni native Americans of New Mexico.” Clin Genet. 49(2):. 95-7.
Khan, T. Z., J. S. Wagener, et al. (1995). “Early pulmonary inflammation in infants with cystic fibrosis.” Am J Respir Crit. Care Med 151(4): 1075-82.
Kiesewetter, S., M. Macek, Jr., et al. (1993). “A mutation in CFTR produces different phenotypes depending on chromosomal background.” Nat Genet. 5(3): 274-8.
Kilinc, M. O., V. N. Ninis, et al. (2000). “Genotype-phenotype correlation in three homozygotes for the cystic fibrosis mutation 2183AA-->G shows a severe phenotype.” J Med Genet. 37(4): 307-9.
Knowles, M. R., N. L. Church, et al. (1990). “A pilot study of aerosolized amiloride for the treatment of lung disease in cystic fibrosis.” N Engl J Med 322(17): 1189-94.
Knowles, M. R., L. L. Clarke, et al. (1991). “Activation by extracellular nucleotides of chloride secretion in the airway epithelia of patients with cystic fibrosis.” N Engl J Med 325(8): 533-8.
Knowles, M. R., K. W. Hohneker, et al. (1995). “A controlled study of adenoviral-vector-mediated gene transfer in the nasal epithelium of patients with cystic fibrosis.” N Engl Med 333(13): 823-31.
Konstan, M. W., P. J. Byard, et al. (1995). “Effect of high-dose ibuprofen in patients with cystic fibrosis.” N Engl J Med 332(13): 848-54.
Kristidis, P., D. Bozon, et al. (1992). “Genetic determination of exocrine pancreatic function in cystic fibrosis.” Am J Hum Genet. 50(6): 1178-84.
Lai, H. C., S. C. FitzSimmons, et al. (2000). “Risk of persistent growth impairment after alternate-day prednisone treatment in children with cystic fibrosis.” N Engl J Med 342(12): 851-9.
Lerer, I., M. Sagi, et al. (1992). “Cystic fibrosis mutations delta F508 and G542X in Jewish patients.” J Med Genet. 29(2): 131-3.
Logan, J., D. Hiestand, et al. (1994). “Cystic fibrosis transmembrane conductance regulator mutations that disrupt nucleotide binding.” J Clin Invest 94(1): 228-36.
Macek, M., Jr., A. Mackova, et al. (1997). “Identification of common cystic fibrosis mutations in African-Americans with cystic fibrosis increases the detection rate to 75%.” Am J Hum Genet. 60(5): 1122-7.
Messiaen, L., C. Van Loon, et al. (1997). “Analysis of 22 cystic fibrosis mutations in 62 patients from the Flanders, Belgium, reveals a high prevalence of Nordic mutation 394delTT.” Hum Mutat 10(3): 236-8.
Mickle, J. E. and G. R. Cutting (2000). “Genotype-phenotype relationships in cystic fibrosis.” Med Clin North Am 84(3): 597-607.
Mittre, H., P. Leymarie, et al. (1995). “High incidence of delta 1507 mutation of the CFTR gene in a limited area of the north west of France.” J Med Genet. 32(7): 577.
Moullier, P., M. Jehanne, et al. (1994). “Association of 1078 del T cystic fibrosis mutation with severe disease.” J Med Genet. 31(2): 159-61.
Murray, J., H. Cuckle, et al. (1999). “Screening for cystic fibrosis.” Health Technol Assess 3(8): i-iv, 1-104.
Noone, P. G., N. Hamblett, et al. (2001). “Safety of aerosolized INS 365 in patients with mild to moderate cystic fibrosis: results of a phase I multi-center study.” Pediatr Pulmonol 32(2): 122-8.
Nunes, V., P. Gasparini, et al. (1991). “Analysis of 14 cystic fibrosis mutations in five south European populations.” Hum Genet. 87(6): 737-8.
Office of Genomics and Disease Prevention, C. (2002). Draft Genetic Test Review: Cystic Fibrosis, Clinical Validity.
Onay, T., O. Topaloglu, et al. (1998). “Analysis of the CFTR gene in Turkish cystic fibrosis patients: identification of three novel mutations (3172de1AC, P1013L and M1028I).” Hum Genet. 102(2): 224-30.
Osborne, L., R. Knight, et al. (1991). “A mutation in the second nucleotide binding fold of the cystic fibrosis gene.” Am J Hum Genet. 48(3): 608-12.
Osborne, L., G. Santis, et al. (1992). “Incidence and expression of the N1303K mutation of the cystic fibrosis (CFTR) gene.” Hum Genet. 89(6): 653-8.
Quan, J. M., H. A. Tiddens, et al. (2001). “A two-year randomized, placebo-controlled trial of domase alfa in young patients with cystic fibrosis with mild lung function abnormalities.” Pediatr 139(6): 813-20.
Ratjen, F. and G. Doring (2003). “Cystic fibrosis.” Lancet 361(9358): 681-9.
Riordan, J. R., N. Alon, et al. (1991). “The CF gene product as a member of a membrane transporter (TM6-NBF) super family.” Adv Exp Med Biol 290: 19-29.
Rolfini, R. and G. Cabrini (1993). “Nonsense mutation R1162X of the cystic fibrosis transmembrane conductance regulator gene does not reduce messenger RNA expression in nasal epithelial tissue.” J Clin Invest 92(6): 2683-7.
Romey, M. C., C. Guittard, et al. (1999). “Complex allele [−102T>A+S549R(T>G)] is associated with milder forms of cystic fibrosis than allele S549R(T>G) alone.” Hum Genet. 105(1-2): 145-50.
Rommens, J. M., M. C. Iannuzzi, et al. (1989). “Identification of the cystic fibrosis gene: chromosome walking and jumping.” Science 245(4922): 1059-65.
Roomans, G. M. (2003). “Pharmacological approaches to correcting the ion transport defect in cystic fibrosis.” Am J Respir Med 2(5): 413-31.
Rozen, R., M. De Braekeleer, et al. (1992). “Cystic fibrosis mutations in French Canadians: three CFTR mutations are relatively frequent in a Quebec population with an elevated incidence of cystic fibrosis.” Am J Med Genet. 42(3): 360-4.
Rubenstein, R. C. and P. L. Zeitlin (1998). “A pilot clinical trial of oral sodium 4-phenylbutyrate (Buphenyl) in deltaF508-homozygous cystic fibrosis patients: partial restoration of nasal epithelial CFTR function.” Am J Respir Crit. Care Med 157(2): 484-90.
Sangiuolo, F., G. Novelli, et al. (1991). “A serine-to-arginine (AGT-to-CGT) mutation in codon 549 of the CFTR gene in an Italian patient with severe cystic fibrosis.” Genomics 9(4): 788-9.
Schaedel, C., L. Hjelte, et al. (1999). “Three common CFTR mutations should be included in a neonatal screening programme for cystic fibrosis in Sweden.” Clin Genet. 56(4): 318-22.
Schibler, A., I. Bolt, et al. (2001). “High morbidity and mortality in cystic fibrosis patients compound heterozygous for 3905insT and deltaF508.” Eur Respir J 17(6): 1181-6.
Schwartz, M., M. Anvret, et al. (1994). “394delTT: a Nordic cystic fibrosis mutation.” Hum Genet. 93(2): 157-61.
Schwarz, M., C. Summers, et al. (1991). “A deletion mutation of the cystic fibrosis transmembrane conductance regulator (CFTR) locus: Delta 1507.” Adv Exp Med Biol 290: 393-8.
Schwarz, M. J., G. M. Malone, et al. (1995). “Cystic fibrosis mutation analysis: report from 22 U.K. regional genetics laboratories.” Hum Mutat 6(4): 326-33.
Schwiebert, E. M., M. E. Egan, et al. (1995). “CFTR regulates outwardly rectifying chloride channels through an autocrine mechanism involving ATP.” Cell 81(7): 1063-73.
Scotet, V., D. Gillet, et al. (2002). “Spatial and temporal distribution of cystic fibrosis and of its mutations in Brittany, France: a retrospective study from 1960.” Hum Genet. 111(3): 247-54.
Sheppard, D. N., D. P. Rich, et al. (1993). “Mutations in CFTR associated with mild-disease-form Cl-channels with altered pore properties.” Nature 362(6416): 160-4.
Shoshani, T., A. Augarten, et al. (1992). “Association of a nonsense mutation (W1282X), the most common mutation in the Ashkenazi Jewish cystic fibrosis patients in Israel, with presentation of severe disease.” Am J Hum Genet. 50(1): 222-8.
Smit, L. S., S. Z. Nasr, et al. (1993). “An African-American cystic fibrosis patient homozygous for a novel frameshift mutation associated with reduced CFTR mRNA levels.” Hum Mutat 2(2): 148-51.
Smith, J. J., S. M. Travis, et al. (1996). “Cystic fibrosis airway epithelia fail to kill bacteria because of abnormal airway surface fluid.” Cell 85(2): 229-36.
Snyder, A. H., M. E. McPherson, et al. (2002). “Acute effects of aerosolized S-nitrosoglutathione in cystic fibrosis.” Am J Respir Crit. Care Med 165(7): 922-6.
Srivastava, M., O. Eidelman, et al. (1999). “Pharmacogenomics of the cystic fibrosis transmembrane conductance regulator (CFTR) and the cystic fibrosis drug CPX using genome microarray analysis.” Mol Med 5(11): 753-67.
Stuhrmann, M., T. Dork, et al. (1997). “Detection of 100% of the CFTR mutations in 63 CF families from Tyrol.” Clin Genet. 52(4): 240-6.
Varon, R., M. Stuhrmarm, et al. (1995). “Pancreatic insufficiency and pulmonary disease in German and Slavic cystic fibrosis patients with the R347P mutation.” Hum Mutat 6(3): 219-25.
Vazquez, C., G. Antinolo, et al. (1996). “Thirteen cystic fibrosis patients, 12 compound heterozygous and one homozygous for the missense mutation G85E: a pancreatic sufficiency/insufficiency mutation with variable clinical presentation.” J Med Genet. 33(10): 820-2.
Vidaud, M., P. Fanen, et al. (1990). “Three point mutations in the CFTR gene in French cystic fibrosis patients: identification by denaturing gradient gel electrophoresis.” Hum Genet. 85(4): 446-9.
Villarreal, M. T., M. Chavez, et al. (1996). “G542X mutation in Mexican cystic fibrosis patients.” Clin Genet. 49(1): 54-6.
Walkowiak, J., K. H. Herzig, et al. (2001). “Analysis of exocrine pancreatic function in cystic fibrosis: one mild CFTR mutation does not exclude pancreatic insufficiency.” Eur J Clin Invest 31(9): 796-801.
Wang, F., S. Zeltwanger, et al. (1998). “Actions of genistein on cystic fibrosis transmembrane conductance regulator channel gating. Evidence for two binding sites with opposite effects.” J Gen Physiol 111(3): 477-90.
Wang, J., M. C. Bowman, et al. (2000). “A novel mutation in the CFTR gene correlates with severe clinical phenotype in seven Hispanic patients.” J Med Genet. 37(3): 215-8.
Wang, M. C., S. G. Shu, et al. (1993). “Cystic fibrosis in two Chinese infants in Taiwan.” Zhonghua Min Guo Xiao Er Ke Yi Xue Hui Za Zhi 34(4): 314-21.
Weinreich, F., P. G. Wood, et al. (1997). “Direct action of genistein on CFTR.” Pflugers Arch 434(4): 484-91.
Will, K., J. Reiss, et al. (1993). “CFTR transcripts are undetectable in lymphocytes and respiratory epithelial cells of a CF patient homozygous for the nonsense mutation R553X.” J Med Genet. 30(10): 833-7.
Wilschanski, M., Y. Yahav, et al. (2003). “Gentamicin-induced correction of CFTR function in patients with cystic fibrosis and CFTR stop mutations.” N Engl J Med 349(15): 1433-41.
Wu, C. L., S. G. Shu, et al. (2000). “Novel cystic fibrosis mutation (2215insG) in two adolescent Taiwanese siblings.” J Formos Med Assoc 99(7): 564-7.
Xiong, X., A. Bragin, et al. (1997). “Structural cues involved in endoplasmic reticulum degradation of G85E and G91R mutant cystic fibrosis transmembrane conductance regulator.” J Clin Invest 100(5): 1079-88.
Yilmaz, E., H. Erdem, et al. (1995). “Study of 12 mutations in Turkish cystic fibrosis patients.” Hum Hered 45(3): 175-7.
Zaman, K., M. McPherson, et al. (2001). “S-nitrosoglutathione increases cystic fibrosis transmembrane regulator maturation.” Biochem Biophys Res Commun 284(1): 65-70.
Zeitlin, P. L., M. Diener-West, et al. (2002). “Evidence of CFTR function in cystic fibrosis after systemic administration of 4-phenylbutyrate.” Mol Ther 6(1): 119-26.
Zielenski, J., D. Bozon, et al. (1991). “Identification of mutations in exons 1 through 8 of the cystic fibrosis transmembrane conductance regulator (CFTR) gene.” Genomics 10(1): 229-35.
Zielenski, J., D. Bozon, et al. (1993). “Analysis of CFTR transcripts in nasal epithelial cells and lymphoblasts of a cystic fibrosis patient with 621+1G-->T and 711+1G-->T mutations.” Hum Mol Genet. 2(6): 683-7.
Zielenski, J., T. M. Fujiwara, et al. (1993). “Identification of the M1101K mutation in the cystic fibrosis transmembrane conductance regulator (CFTR) gene and complete detection of cystic fibrosis mutations in the Hutterite population.” Am J Hum Genet. 52(3): 609-15.
Zielenski, J., D. Markiewicz, et al. (1995). “Skipping of exon 12 as a consequence of a point mutation (1898+5G-->T) in the cystic fibrosis transmembrane conductance regulator gene found in a consanguineous Chinese family.” Clin Genet. 47(3): 125-32.
Zielenski, J. and L. C. Tsui (1995). “Cystic fibrosis: genotypic and phenotypic variations.” Annu Rev Genet. 29: 777-807.

What is claimed is:

1. A method for detecting in a nucleic acid sample the presence or absence of two or more mutations at selected mutation sites associated with cystic fibrosis, the method comprising the steps of;
a) amplifying from the sample regions of DNA containing the two or more selected mutation sites to form amplified DNA products;
b) hybridizing at least two tagged allele specific extension primers to a complementary target sequence in the amplified DNA products, wherein each tagged allele specific extension primer has a 3′-end hybridizing portion substantially complementary to a portion of the amplified DNA product and a 5′-end tag portion complementary to a corresponding anti-tag sequence, the 3′-end hybridizing portion having a 3′ terminal nucleotide that is either complementary to a suspected variant nucleotide or to the corresponding wild type nucleotide of one of the selected mutation sites;
c) extending those at least two tagged allele specific extension primers whose 3′ terminal nucleotides are perfectly complementary to an allele of one of the selected mutation sites, using labelled nucleotides; and
d) hybridizing the at least two tagged allele specific extension primers to their corresponding anti-tag sequences and detecting the presence of labelled extension products.

2. The method of claim 1 wherein the 3′ end hybridizing portions of the at least two tagged allele specific extension primers each comprise a sequence selected from the group consisting of nucleotides from position 25 to the 3′ terminal nucleotide of SEQ ID NO: 33 to SEQ ID NO: 118 and wherein the 3′ end hybridizing portion of each of the at least two tagged allele specific extension primers has a different sequence.

3. The method of claim 2 wherein the 5′-end tag portions of the at least two tagged allele specific primers each comprise a sequence selected from the group consisting of nucleotides from position 1 to 24 of SEQ ID NO: 33 to SEQ ID NO: 118 and wherein the 5′-end tag portion of each of the at least two tagged allele specific primers has a different sequence.

4. The method of claim 2 wherein the at least two tagged allele-specific extension primers each comprise a sequence selected from the group of sequences consisting of SEQ ID NO: 33 to SEQ ID NO: 118.

5. The method of claim 1 wherein step (a) is conducted by PCR.

6. The method of claim 1 wherein the anti-tag sequence is coupled to a solid support.

7. (canceled)

8. The method of claim 5 wherein the step of PCR amplifying is conducted using PCR amplification primers selected from the group of primer pairs consisting of:
SEQ ID NO.: 11 and SEQ ID NO.: 12, SEQ ID NO.: 13 and SEQ ID NO.: 14, SEQ ID NO.: 15 and SEQ ID NO.: 16, SEQ ID NO.: 1 and SEQ ID NO.: 2, SEQ ID NO.: 17 and SEQ ID NO.: 18, SEQ ID NO.: 3 and SEQ ID NO.: 4, SEQ ID NO.: 19 and SEQ ID NO.: 20, SEQ ID NO.: 21 and SEQ ID NO.: 22, SEQ ID NO.: 25 and SEQ ID NO.: 26, SEQ ID NO.: 5 and SEQ ID NO.: 6, SEQ ID NO.: 7 and SEQ ID NO.: 8, SEQ ID NO.: 27 and SEQ ID NO.: 28, SEQ ID NO.: 29 and SEQ ID NO.: 30, SEQ ID NO.: 9 and SEQ ID NO.: 10, and SEQ ID NO.: 23 and SEQ ID NO.: 24, wherein the primer pairs are selected for their ability to amplify regions of DNA that include sequences to which the selected at least two tagged allele-specific extension primers will hybridize.

9. A kit for use in detecting in a nucleic acid sample the presence or absence of a variant nucleotide in at least two selected mutation sites known to be associated with cystic fibrosis, said kit comprising a set of at least two tagged allele specific extension primers wherein each tagged allele specific extension primer has a 3′-end hybridizing portion having a 3′ terminal nucleotide perfectly complementary to an allele of one of the selected mutation sites—and a 5′-end tag portion complementary to one of a set of anti-tags.

10. The kit of claim 9 wherein the 3′ end hybridizing portions of the at least two tagged allele specific extension primers each comprise a sequence selected from the group consisting of nucleotides from position 25 to the 3′ terminal nucleotide of SEQ ID NO: 33 to SEQ ID NO: 118 and wherein the 3′ end hybridizing portion of each of the at least two tagged allele specific extension primers has a different sequence.

11. The kit of claim 9 wherein the 5′-end tag portions of the at least two tagged allele specific primers each comprise a sequence selected from the group of sequences consisting of nucleotides from position 1 to 24 of SEQ ID NO: 33 to SEQ ID NO: 118 and wherein the 5′-end tag portion of each of the at least two tagged allele specific primers has a different sequence.

12. The kit of claim 10 wherein the at least two tagged allele-specific extension primers comprise sequences selected from the group of sequences consisting of SEQ ID NO: 33 to SEQ ID NO: 118.

13. The kit of claim 9 further comprising a set of PCR amplification primers selected from the group of primer pairs consisting of:
SEQ ID NO.: 11 and SEQ ID NO.: 12, SEQ ID NO.: 13 and SEQ ID NO.: 14, SEQ ID NO.: 15 and SEQ ID NO.: 16, SEQ ID NO.: 1 and SEQ ID NO.: 2, SEQ ID NO.: 17 and SEQ ID NO.: 18, SEQ ID NO.: 3 and SEQ ID NO.: 4, SEQ ID NO.: 19 and SEQ ID NO.: 20, SEQ ID NO.: 21 and SEQ ID NO.: 22, SEQ ID NO.: 25 and SEQ ID NO.: 26, SEQ ID NO.: 5 and SEQ ID NO.: 6, SEQ ID NO.: 7 and SEQ ID NO.: 8, SEQ ID NO.: 27 and SEQ ID NO.: 28, SEQ ID NO.: 29 and SEQ ID NO.: 30, SEQ ID NO.: 9 and SEQ ID NO.: 10, and SEQ ID NO.: 23 and SEQ ID NO.: 24, wherein the primer pairs are selected for their ability to amplify regions of DNA that include sequences to which the selected at least two tagged allele-specific extension primers will hybridize.

14. The kit of claim 10 further comprising a set of anti-tags, each anti-tag having a sequence complementary to nucleotides 1 to 24 of the selected at least two tagged allele-specific extension primers.

15. The kit of claim 14 wherein the anti-tags are coupled to a support.

16-17. (canceled)

18. A composition comprising a plurality of polynucleotide primers for use in detecting the presence or absence of variant nucleotides associated with cystic fibrosis, wherein the plurality of primers comprises oligonucleotides having sequences set forth by nucleotides from position 25 to the 3′ terminal nucleotide of SEQ ID NO: 33 to SEQ ID NO: 118, or the complete complements thereof.

19. The composition of claim 18 wherein the plurality of primers consists of oligonucleotides having sequences set forth by SEQ ID NOs: 33-118, or the complete complements thereof.

20. A combination comprising the composition of claim 19 wherein the plurality of primers consists of oligonucleotides having sequences set forth by SEQ ID NOs: 33-118, and a set of anti-tags, the set of anti-tags having sequences complementary to nucleotides 1-24 of SEQ ID NOs: 33-118.

21. A combination comprising the composition of claim 19 wherein the plurality of primers consists of oligonucleotides having sequences set forth by the complete complements of SEQ ID NOs: 33-118, and a set of anti-tags, the set of anti-tags having sequences complementary to nucleotides 1-24 of the complete complements of SEQ ID NOs: 33-118.

22. The combination of claim 20, wherein each anti-tag is attached to a spectrally coded bead specific for the anti-tag.

23. The combination of claim 21, wherein each anti-tag is attached to a spectrally coded bead specific for the anti-tag.

24. An improved method of simultaneously detecting in a sample the presence or absence of variant nucleotides associated with cystic fibrosis, where in the improvement comprises simultaneously identifying the presence or absence of variant nucleotides associated with cystic fibrosis via allele-specific primer extension using a set of primers having the sequences set forth in SEQ ID NOs: 33-118.

25. The method of claim 1, wherein each tagged allele specific extension primer comprises a 3′ end hybridizing portion having a 3′ terminal nucleotide that is perfectly complementary to a suspected variant nucleotide associated with cystic fibrosis.

26. The method of claim 25, wherein for each tagged allele specific extension primer comprising a 3′ end hybridizing portion having a 3′ terminal nucleotide that is perfectly complementary to a suspected variant nucleotide at the selected mutation site, there is a tagged allele specific primer whose 3′ end hybridizing portion has a 3′ terminal nucleotide that is perfectly complementary to the corresponding wild type nucleotide at the selected mutation site.

27. The kit of claim 10, wherein the at least two tagged allele specific extension primers comprise sequences each having a 3′ end terminal nucleotide that is perfectly complementary to a variant nucleotide known to be associated with cystic fibrosis.

28. The kit of claim 27, wherein for each of the at least two tagged allele specific extension primers comprising a sequence having a 3′ end terminal nucleotide that is perfectly complementary to a variant nucleotide known to be associated with cystic fibrosis, there is a corresponding tagged allele specific extension primer comprising a sequence having a 3′ end terminal nucleotide that is perfectly complementary to a corresponding wild-type nucleotide.
